(19) United States
(12) Patent Application Publication
(10) Pub. No.: US 2026/0021233 A1
(43) Pub. Date: Jan. 22, 2026

(54) A HIGH PERFORMANCE IMMUNOAFFINITY BASED SYSTEM FOR EXTRACORPOREAL CAPTURE OF PATHOGENS, CANCER CELLS AND TOXINS FROM BLOOD

(71) Applicants: PANNON EGYETEM, Veszprém (HU); CAPTEC MEDICAL KFT., Budapest (HU)

(72) Inventors: András GUTTMAN, Budapest (HU); Gábor JÁRVÁS, Veszprém (HU); Paul Anton STOLK, Budapest (HU); József TÓVÁRI, Budapest (HU); László Kristóf TAKÁCS, Budapest (HU)

(21) Appl. No.: 18/875,423

(22) PCT Filed: Jun. 15, 2023
(86) PCT No.: PCT/HU2023/050037

§ 371 Date: Dec. 16, 2024

(30) Foreign Application Priority Data
Jun. 15, 2022 (HU) P2200216

(51) Int. Cl. A61M 1/34; A61M 60/109; A61M 60/279; A61M 60/37

(52) U.S. Cl. CPC A61M 1/34; A61M 60/109; A61M 60/279; A61M 60/37; A61M 2205/3334; A61M 2205/7509; A61M 2205/7545

(57) ABSTRACT

The invention relates to an immuno-affinity cell capture system comprising a cartridge or column which contains a high amount of activated beads of a specific diameter having binding agents against specific cells in the blood, e.g. pathogenic cells or viruses, in a housing equipped with appropriate fittings to allow perfusion of blood whereby said cells from blood bind to the beads. Thereby removal of said cells from blood and thereby cleansing of blood becomes possible. Also the beads can preferably glass beads in the range of 300 to 500 μm. This method allows binding a very high number of cells and has a very high capacity and a highly versatile system to adapt to various different pathogens.

### FIELD OF THE INVENTION

The invention relates to an immuno-affinity cell capture system comprising a cartridge or column which contains a high amount of activated beads of a specific diameter having binding agents against specific cells in the blood, e.g. pathogenic cells or viruses, in a housing equipped with appropriate fittings to allow perfusion of blood whereby said cells from blood bind to the beads.

Thereby removal of said cells from blood and thereby cleansing of blood becomes possible.

Also the beads can preferably glass beads in the range of 300 to 500 μm. This method allows binding a very high number of cells and has a very high capacity and a highly versatile system to adapt to various different pathogens.

### BACKGROUND ART

By removing CTCs from the bloodstream, development of metastases could be prevented. These methods may be based on technologies such as immuno-capture with specific antibodies covalently bound to the surface of the device [Gaitas, A. and G. Kim 2015] [22], non-specific capture [Kang, J. H., et al. 2014] [23], or photo-immunotherapy [Kim, G. and A. Gaitas, 2015]. Extracorporeal photopheresis (ECP) can be applied in case of leukemia and T-cell lymphoma [Garban, F., et al. 2012: Vieyra-Garcia, P. A. and P. Wolf 2020] [25, 26]. During ECP treatment, the leukocytes are exposed to 8-methoxypsoralen photosensitizer and ultraviolet-A light. Nonetheless, the existing procedure has limited selectivity and efficiency and produces partial response in the majority of treated patients, expensive and takes a very long time [Darvekar, S., et al. 2020] [27]. Another interesting modality has been proposed by Gaitas and Kim [Gaitas, A. and G. Kim 2015] [22]. They examined the cell-capture efficiency of the immune-activated inner wall of a PDMS polymer tube. This technic also has been investigated for not just CTCs, but bacteria removal [Kim, G., H. Vinerean, and A. Gaitas, 2017] [28]. Modification of hemodialysis membranes could also be an option for high volume CTC removal therapy [Jarvas, G., et al. 2021] [29].

Next to the hemofilters, hemoperfusion and plasmapheresis procedures are used to non-specific pathogen removal from the bloodstream such as heparin immobilized polyethylene beads. The end-point attached heparin binds to viruses, bacteria, fungi and toxins similarly to heparane-sulfate interacting with cell-surface [Seffer, M. T., et al. 2021: Pape, A., et al. 2021] [38, 39]. Hemopurifirer lectin affinity plasmapheresis filters have also been designed for whole virus, exosome and exosomal microRNA removal and investigated for COVID-19 therapy [Amundson, D. E., et al. 2021] [40].

Thus, extracorporeal hemopurification methods are known in the art. However, such methods, if utilize pathogen-recognition by capturing molecules, are usually highly specific and thus limited to a given pathogen.

US20150283318A1 (Methods to detect and treat diseases) provides a method to treat pathogen infection by inactivating the pathogens in the blood. During the treatment, blood is withdrawn from a patient and is separated into its plasma and cellular components. The invention also provides a method to treat cancer especially to prevent tumor metastasis and tumor recurrence by removing and/or inactivating (e.g. killing) the circulating tumor cells (CTC) in the blood after removing the tumor or treating the tumor with therapeutical means. In an example (example 9), to perform CTC removal, 300 um diameter Sephadex beads coated with antibody against CTC surface marker is added to the extracorporeally circulating blood and then the blood is passing through a filter having pore size of 200 μm to remove the beads as well as beads bound CTC before the blood goes back to the patient. Glass beads are mentioned as possible alternatives. The circulating tumor cells are removed by passing the extracorporeally circulating blood through a circulating tumor cell removal device.

Bankó, P. et al. [Bankó, P. et al. (2019). Technologies for circulating tumor cell separation from whole blood. Journal of hematology & oncology, 12 (1), 48. https://doi.org/10.1186/s13045-019-0735-4] summarize the available CTC technologies. The hemoperfusion is not mentioned in the article.

The possibility of removing CTCs and viruses require specific methods according to the art.

This method is more rarely applied with other pathogens or toxins, however, certain methods are known.

EP2533828 provide a method for the removal from mammalian blood of a toxin from a pathogen; in an embodiment the invention is to provide an extracorporeal treatment of mammalian blood, in conjunction with a therapy for treating diseases caused by Bacillus anthracis, Pseudomonas aeruginosa or Staphylococcus aureus by removing the pathogens or toxins from the pathogens from mammalian blood by contacting mammalian blood with a solid essentially nonporous substrate. Said substrate may comprise a packed column of non-porous rigid beads or particles or a column packed with a rigid monolith bed of sintered beads with internal flow channels. The size of the interstitial channel and other conditions may be such that when said sample of blood is in flow contact with said substrate at a flow rate of ≥50 ml/min, said toxin binds to said heparin; in a particular embodiment the flow rate of blood or serum ranges from about 150 and 2000 mL/minute (which should be converted into linear rate to be comparable) and said beads have a diameter ranging from 100 to 450 microns, such as an average diameter of 0.3 mm.

Apparently, there is still a need to develop a versatile platform which is readily adaptable to a plurality of pathogenic materials.

In the present invention the idea of hemoperfusion and immune-affinity systems have been combined and further developed. The present inventors have designed a hemoperfusion system which proved to be surprisingly feasible.

The object of the present invention has been to develop a flow capture device to remove pathogens, e.g. cells or virions from human cardiovascular circulation thereby preventing the development of serious clinical outcome and a system to apply it to different pathogens. The approach combines the specificity of affinity chromatography with the high throughput hemoperfusion technique.

### BRIEF DESCRIPTION OF THE INVENTION

The invention relates to a kit of parts for assembly of a device for extracorporeal capturing of pathogenic material from a patient's blood thereby cleansing the patient's blood.

### In Particular, the Invention Relates to the Kit of Parts Comprising

beads, each bead having the diameter of 300 μm to 500 μm, preferably 350 μm to 450 μm and having or being suitable to have, attached to their surface, capturing molecules to capture (capable of binding) a pathogenic material,
a fillable cartridge, said cartridge having a first opening and a second opening and a first mesh and a second mesh to withhold the beads within the inner space of the cartridge,
means to allow the filling of the cartridge with beads.

### In a First Preferred Embodiment the Invention Relates to a Kit of Parts Comprising

a set of one or more (preferably at least two) multitudes of beads, each bead having the diameter of 300 μm to 500 μm, preferably 350 μm to 450 μm and having, attached to their surface, capturing molecules to bind (capable of binding) a pathogenic material, wherein beads of a multitude comprises beads having capturing molecules for the same pathogenic material, and different multitudes comprise beads having binding molecules for the different pathogenic materials
a fillable cartridge, said cartridge having a first opening and a second opening and a first mesh and a second mesh to withhold the beads within the inner space of the cartridge,
means to allow the filling of the cartridge with beads.

Preferably the kit comprises at least two multitudes of beads. Thereby the device assembled from the kit of parts is suitable for capturing the pathogens of interest from a subject blood.

### In a Second Preferred Embodiment of the Kit, Said Kit Comprises at Least Two

a multitude of beads, each bead having the diameter of 300 μm to 500 μm, preferably 350 μm to 450 μm and having, attached to their surface, capturing molecules to bind (capable of binding) a pathogenic material,
a fillable cartridge, said cartridge having a first opening and a second opening and a first mesh and a second mesh to withhold the beads within the inner space of the cartridge,
means to allow the filling of the cartridge with beads,
a set of one or more (preferably at least two) multitudes (populations?) of binding molecules for binding pathogenic material, wherein said binding molecules are useful to be attached to the surface of the beads,
wherein capturing molecules of a multitude (population) comprises beads having capturing molecules for the same pathogenic material, and different multitudes (populations) comprise beads having capturing molecules for the different pathogenic materials.

The diameter of the beads is preferably 320 μm to 480 μm, preferably 340 μm to 460 μm, preferably 350 μm to 450 μm, highly preferably 370 μm to 430 μm. As shown by the present inventors the size range of the beads is in relation to the effectiveness of the extracorporeal blood cleansing method. A higher diameter would result in a smaller specific surface area and therefore binding capacity whereas a smaller diameter results in higher hydrodynamic resistance and pressure drop which either increase shear force if flow velocity is maintained or at lower shear force the flow velocity is low. This parameter allows a very versatile and robust method applicable to multiple pathogen and subject thereby leading to the system of the invention and allowing using it as a general platform for blood cleansing in a broad range of medical treatments.

In a particular embodiment there are at least 2, preferably at least 3, 4, 5, 6, 7, 9 or 10, preferably at least 10, 20, 30, 50 or 100 multitudes of bead or capturing molecules.

In this second embodiment preferably the set of one or more, preferably at least two, preferably more multitudes (or populations) of capturing molecules is (are) suitable for the preparation of a set of one or more (preferably at least two) multitudes (or populations) of beads.

In a particular embodiment the kit of parts comprises a composition or a reagent kit for attaching the capturing molecules to the beads.

The kit or parts is useful to prepare a device comprising the cartridge filled with the beads

Preferably, said composition or reagent kit comprises a derivatization agent.

Preferably, said composition or reagent kit comprises a linker.

Optionally, said composition or reagent kit comprises appropriate buffer, reagent, solvent etc. to carry out the attachment process.

Preferably in the embodiments of the invention the cartridge has opening (1) for the perfusion of blood. Preferably the cartridge has a first opening (1a) and a second opening (1b). In an embodiment blood enters the cartridge through the first opening (in this case inlet or inlet opening) and leaves the cartridge via the second opening (1b) (in this case outlet or outlet opening).

In a preferred embodiment the direction of the blood flow defines the inlet and outlet openings. In an embodiment the

The cartridge also comprises a mesh for withholding the beads. Preferably the cartridge comprises a first mesh (2a) and a second mesh (2b). In an embodiment the mesh (2) is placed in the opening. The mesh (2) may be removable or fixed. If removable it should be fastened or positioned.

The cartridge also has a means to fill the cartridge, in particular useful space (6) of the cartridge which contains or is formed to contain the beads. The means to fill the cartridge may be the opening (1) provided that the mesh is removable and can be fixed separately into the opening.

In a preferred embodiment the means to fill the cartridge is a removable lid e.g. lid (5) which can be fastened to the wall of the cartridge by a known means e.g. through a spiral or screw. In such cases preferably an O-ring is applied between the lid and the wall of the cartridge.

The cartridge can be connected to a tubing e.g. tubing (50) for blood flow. Blood flow can be driven by a pump, e.g. a peristaltic pump (10).

The volume of the inner space of the cartridge is set to provide a sufficient amount of binding site (capturing site), wherein preferably the number of the specific binding sites is at least 50,000 per ml, preferably at least 70,000 per ml or at least preferably 100,000, in highly preferred embodiments at least 0.5×10⁶ or at least 1.0×10⁶ or at least 1.5×10⁶.

Highly preferably, the system or kit according to the invention is useful to prepare a device wherein the specific binding sites is at least 30,000 per ml, preferably at least 40,000 per ml or at least preferably 50,000 per ml.

Preferably the beads are non-porous beads.

The beads have capture molecules on their surface to capture pathogenic material.

The pathogenic material can be a pathogenic agent which can be cellular pathogen, including cancer cells or a microorganism, extracellular vesicles or can be a virus. In alternative embodiment the pathogenic material can be a toxin.

A toxin may be a protein toxin of a pathogen, e.g. a pathogenic microorganism or bacterium.

A toxin also can be an endotoxin. In case of certain pathogens toxins may be provided in extracellular vesicles. In such cases it is advantageous if the toxin is present in the membrane and can be recognized by the capturing molecule.

Capturing molecules include antibody, proteins of antibody derived protein scaffolds, like fragments, single-domain antibodies, single chain antibody fragments (scFv), Fab fragments or nanobodies, aptamers.

Alternatively, a capture molecule can be entirely artificially made, e.g. as a synthetic peptide, proteins of non-antibody protein scaffolds like fibronectin, lipocalins, anticalins or affibodies, nucleic acids, etc.

Preferably the cartridge comprises multiple types of beads i.e. beads with single or multiple capturing molecules against multiple pathogenic materials (antigens).

In an embodiment therefore the cartridge can be designed against a specific disease wherein multiple antigenic epitopes are targeted. For example in case of cancer the EPCAM antibody is of a broad spectrum while also more specific antigens like the CD44 in case of colon cancer (to give an example), can be targeted.

Preferably the volume of the cartridge is at least 4 ml per liter of total blood volume of the subject.

In a preferred embodiment the useful space is at least 20 ml in case of a human subject, preferably an adult human subject.

Preferably, the cartridge having a volume of its useful space (i.e., wherein the beads are placed) of 20 to 500 ml, preferably 100 to 500 ml, preferably 300 to 400 ml in case of an adult human subject, preferably an adult human subject.

The shape of the cartridge should allow laminar flow and the sufficient permeability.

As to the design of the cartridge and amount of the beads and related principles see the detailed description.

Said fillable cartridge is useful for making blood flowing through said cartridge. Said fillable cartridge has a blood inlet and a blood outlet and has an outlet mesh and an inlet mesh to withhold the beads. The pore size (mesh size) of the mesh is smaller than the diameter of the beads, or if there are beads with multiple diameters smaller than the smallest diameter to withhold the beads. Preferably the pore size of the mesh is at most 70%, preferably at most 80%, preferably at most 90% or the diameter of the beads having the smallest diameter in the cartridge.

A mesh as used herein is a separating wall with pores having a pore size smaller than the lowest diameter of the beads so to withhold the entirety of the beads within the cartridge. Preferably both the first (inlet) mesh and a second (outlet) mesh are applied. Preferably both meshes have the same properties and thus the direction of the flow is reversible.

In a preferred embodiment the kit also comprises tubing for making blood flowing through.

Preferably, the system or kit also comprises derivatization agents to elaborate capturing of the capturing molecules to the beads. Such techniques are known in the art.

Preferably the surface of said beads is or can be covered by a substance reducing non-specific capturing of the pathogens. In a preferred embodiment such non-specific binding reducer also forms part of the system or kit.

### Preferably, the System or Kit According to the Invention is Useful to Prepare a Device Comprising

beads having the diameter of 300 μm to 500 μm, preferably 350 μm to 450 μm and having a surface which is or can be derivatized to attach capture molecules to capture the pathogenic material to said beads,
a fillable cartridge, said cartridge having an inlet and an outlet and a first (or an inlet) mesh and a second (or an outlet) mesh to withhold the beads within the inner space of the cartridge
one or more preparation(s) of capturing molecules against (i.e. capable of capturing of) one or more pathogenic material.

In particular the cartridge is useful to specifically capture at least 30,000 cells per ml, preferably at least 40,000 cells per ml or at least preferably 50,000 cells per ml.

A particularly preferred cartridge of the invention has a useful volume (i.e. a volume of the suspension of beads of 300 to 500 ml which means that the device is capable of capturing (has a capacity of), in case a relatively smaller cartridge, at least 0.9×10⁶ binding sites, preferably at least 1.2×10⁶ binding sites, more preferably at least 1,5×10⁶ binding sites, or in case a relatively larger cartridge (e.g. of 500 ml), at least 1.5×10⁶ binding sites, preferably at least 2×10⁶ binding sites, more preferably at least 2.5×10⁶ binding sites.

In a further embodiment the invention relates to a system, which also comprises means to operate the device.

The elements of the system are the device according to the invention and further means to operate the device to extracorporeal cleansing of blood.

In an embodiment the elements of the system are the kit of parts according to any embodiment according to the invention and further means to operate the device to extracorporeal cleansing of blood.

According to an embodiment of the invention the system comprises a pump and a control unit to set flow rate as means to operate the device. In particular the control unit able to operate the pump and thereby regulate flow velocity.

In a further embodiment of the invention the system comprises a means for preventing blood from bubble formation, e.g. a bubble trap.

In a preferred embodiment the pump can be operated from a microprocessor or a computer connected or being part of the control unit and programmed to set the flow rate and maintain it within limits.

In a preferred embodiment the control unit regulates or controls the blood flow, including flow velocity

In a further embodiment the system comprises means to measure certain parameters of the effluent, cleansed blood, for example the level of the pathogenic material can be monitored. Preferably these data are provided to the control unit which sets the operation parameters based on these data. More specific embodiments are described in the detailed description.

The flow rate may be calculated per ml of the device cartridge as about 2 ml/min per cm³ of the cartridge volume or in a broader range a 1 to 3 ml/min per cm³ or 1.5 to 2.5 ml/min per cm³. Thus, in case of an about 2.5 cm³ cartridge (i.e. a cartridge containing about 2.5 cm³ beads of 400 μm (or size of 350 μm to 500 μm, to give a broader range) an advisable volume flow rate is ab 2.5 to 7.5 ml/min, or preferably 3.75 to 6.25 ml/min, in particular about 5 ml/min.

In case of a 300 ml cartridge the advisable flow rate is in the range of 300 to 900 ml/min, or 400 to 700 ml/min or about 500 or 600 ml/min.

In a preferred embodiment the linear flow rate is preferably between 12 to 600 cm/min, preferably 12 to 300 cm/min, highly preferably 12 to 120 cm/min or 24 to 600 cm/min, preferably 24 to 300 cm/min, highly preferably 24 to 150 cm/min or 60 to 600 cm/min, preferably 60 to 300 cm/min, highly preferably 60 to 150 cm/min. (60 cm/min is equal to 1 cm/s or 0.01 m/s).

In a particularly preferred embodiment the linear flow rate is 24 to 150 cm/min.

These ranges typically provide an effective capturing still a low shear rate.

The shape of the cartridge should allow or help laminar flow in the appropriate range to arrive at a robust solution. Preferably the shape of the cartridge is elongated, however, has a sufficient diameter and a low void (dead) volume wherein there are no beads.

In a typical embodiment the length of the cartridge is 0.5 to 10, preferably 0.5 to 5 or 1 to 5, preferably 1 to 4 times that of the diameter of the cartridge.

In a preferred embodiment the length of the cartridge is 1.5 to 2, or 1.5 to 2.5 or 2 to 3 times that of the diameter of the cartridge.

In certain embodiments, if the length of the cartridge is about the same or shorter than its diameter then preferably a fluid dispenser is applied to arrive at an even distribution of flow rates (or essentially identical flow rate) throughout the cartridge.

In an example the method can be carried out in a commercially available hemoperfusion cartridge. In an example the inner diameter of the cartridge is 5.5 cm while the effective length is 12 cm. Applying this geometry arrangement, the typical linear velocity is around 0.025 m/s.

In a particular embodiment the blood of the subject flows through the device wherein the flow velocity can be set and regulated as describe above.

The material of the device is a biocompatible material, preferably a biocompatible plastic e.g. polycarbonate or other plastic material.

The material of the beads may be any plastic material which can be derivatized to capture a protein type capturing molecule, said plastic material including but not limited to polypropylene, PET, polycarbonate, PMMA, PDMS, HD/LD-PE. In alternative embodiment the beads can be prepared of e.g. polystyrene, polysulfone, etc.

The bead also can be made of silica.

The sealing may be made of e.g. silicone or other inert and resilient material.

The mesh that withholds the beads may be e.g. of polyester or polypropylene.

In an embodiment the capturing molecule is selected from a group consisting of proteins with specific capturing site(s), in particular antibodies, glycoproteins, in particular mucins and/or lectins, oligonucleotide capturing agents like aptamers, small capturing molecules and ligands, in particular folic acid and any combinations of thereof.

In a preferred embodiment the capture molecule is a protein-type capturing molecule e.g. an antibody, a nanobody, a single-domain antibody etc.

In a particular embodiment the capturing agent is an antibody or a binding fragment thereof or a biomolecule having a binding region of an antibody, said antibody being preferably a tumor specific antibody, in particular an antibody adapted to said mammal, more preferably an antibody selected from the group of anti-CD44 and anti-EpCAM antibodies.

### The Invention Also Relates to a Method for Cleansing Blood, Said Method Comprising

allowing blood of a patient to flow through the device as described herein,
leading the cleansed blood back to the patient.

The method is a method of treatment wherein the pathogens are captured by the device of the invention from the blood of the patient.

In a preferred embodiment the device is operated in the system of the invention, e.g. the flow rate is regulated as described above.

The pathogen may be any pathogenic material as described herein. The disease can be any disease caused by any pathogen as described herein, in particular a cancer, a viral disease, a disease caused by a toxin etc.

As used herein the singular forms “a”, “an” and if context allows “the” include plural forms as well unless the context dictates otherwise.

The term “comprises” or “comprising” (being equivalent to and replaceable by “including”) are to be construed herein as having a non-exhaustive meaning and allow the addition or involvement of further features or method steps or components to anything which comprises the listed features or method steps or components. The term “comprises” and equivalents can be limited e.g. to “consisting essentially of” or “comprising substantially” understood as consisting of mandatory features or method steps or components listed in a list e.g. in a claim whereas allowing to contain additionally other features or method steps or components which do not materially affect the essential characteristics of the use, method, composition or other subject matter.

### BRIEF DESCRIPTION OF THE FIGURES

FIG. 1A The laboratory scale system for viruses with the test cartridge (Panel A) and its schematic representation (Panel B). The circulation was supported by a peristaltic pump (10). The parts of the system-cartridge (4), vessel (30) are connected with polyethylene tubing (50). The virus suspension in the vessel was homogenized by a magnetic stirrer (40).

FIG. 1B The laboratory scale system for CTCs with the test cartridge (Panel A) and its schematic representation (Panel B). The circulation was maintained by a LeadFluid peristaltic pump (10). The laboratory scale cartridge (4) was filled with activated/control beads, and the tank were filled with the cell-buffer suspension-symbolizing the human body. The even concentration of the cells was ensured by magnetic stirrer (40). Parts of the test system were connected by polyethylene tubing.

FIG. 2A Schematic representation of the virus capture mechanism. The immobilized nanobodies specifically bound the spike protein of SARS-COV-2 particles, thus, selectively capture them from the blood stream. Drawing is not for scale.

FIG. 2B Schematic of the capture mechanism for CTCs. The beads selectively capture CTCs, while blood components pass through the cartridge. Drawing is not for scale.

FIG. 3. Scanning electron microscope (SEM) images of the virions. Panel A-B: virus suspension dried onto a microscope side-grid. Panel C-D: the captured virus particles on the surface of the beads. Images were taken using a FEI Quanta 3D FEG instrument.

FIG. 4 Histograms of the flow cytometry measurement of FITC-labelled anti-EpCAM conjugation. Panel A: Unconjugated cells gated in the D region did not emit green fluorescence. Panel B: FITC labelled cells gated in the E region show definite green fluorescence after conjugation

FIG. 5 Contact angle measurements were carried out to investigate the quality of the prepared glass surface. As a result of the chemical cleaning, the number of hydroxyl groups on the surface increased, resulting in a spectacular decrease in the contact angle. Subsequently, due to the hydrophobic property formed by the APTES molecules bound to the surface functional group, the contact angle increases

FIG. 6 Results of the capture experiments. Panel A: Increasing flow rate reduces non-specific capturing, thus higher specific capture could be achieved. Panel B: The change in bead size, i.e. the increase of active surface area had a positive effect on specific capture, which increased by about 74%. Panel C-D: Anticoagulation experiments confirmed that the use of Na-Citrate and LMWH does not affect the function of the beads adversely and these anticoagulant agents can be used in the future during treatments.

FIG. 7 Fluorescent images of activated beads and captured cells on different glass surfaces. Panels A-B: Activated beads (B) show uniform fluorescence after successful FITC-conjugated anti-EpCAM immobilization, unlike control beads (A) containing only linkers. Panels C-D: Difference in cell capture efficiency between active (D) and control (C) glass slides. Panel E: Captured cells on the surface of activated beads. Images were taken using a Nikon Eclipse Ni fluorescence microscope equipped with a FITC (ex.: 475 nm/em.: 530 nm) band-pass filter cube.

FIG. 8 Scheme of the proposed immobilization methods. A.: Amination of cleaned native PMMA with hexamethylenediamine at basic pH and cross-linking with glutaraldehyde. B: Silanization of oxidized PMMA surface with APTES and cross-linking with glutaraldehyde. Oxidation of the PMMA surface can be carried out with acids or other oxidizing agents under vigorous conditions. Immobilization of the protein of choice is completed by Schiff's base reaction.

FIG. 9 Blocking of excess reactive aldehyde groups by reversible PEG adsorption.

FIG. 10 An exemplary cartridge in two versions wherein the two directions are equivalent or not. 1 inlet 1a blood inlet 1b blood outlet; 2 mesh 2a first mesh 2b second mesh; 3 beads; 4 cartridge; 5 lid; 6 useful space

FIG. 11 Shows the design of a system comprising cartridge 4 of the invention. The system comprises a peristaltic pump and a control unit as described herein.

### DETAILED DESCRIPTION OF THE INVENTION

The present inventors have implemented a novel approach to remove pathogenic material from a patient's blood.

In the present invention a high throughput extracorporeal device has been designed to specifically remove harmful biological material, e.g. pathogens, including viral or cellular pathogens, harmful cells or toxins from the cardiovascular circulation. The object of the method is to reduce the level of harmful biological material in the patient's blood and thereby avoid or prevent a serious clinical outcome.

The blood purification device is part of a hemoperfusion system developed by the present inventors offers efficient capture and removal of the targeted pathogen from human cardiovascular circulation, thus decreasing its load.

The device or its parts is/are element(s) of a flexible system which can quickly respond the needs of patients having a pathogen in their blood.

In an aspect the invention is a versatile system or kit for the manufacture of a device for use in an extracorporeal method for removal of pathogenic material from a patient's blood.

The system or kit is quickly adaptable to a specific pathogenic material.

For example it is quickly adaptable to the need of a subject (patient) or group of subjects having a specific pathogenic material in his/her/their blood. Once the pathogenic material is diagnosed the extracorporeal blood purification? device of the invention can be produced in a short time.

In an embodiment the system can be used in case of an epidemic or pandemic caused by a known causative agent, as a pathogenic material, i.e. pathogenic agent. Once the causative agent is identified and reported, the devices against said causative agent can be manufactured readily and shipped within a very short time from such reporting and effective treatment by blood cleansing can be started in patients diagnosed with said pathogenic agent in e.g. local healthcare units.

In case of known pathogenic materials the capturing molecules can be stored and/or ordered and shipped as a part of the system or kit. Thus, in case of expectable outbreaks, for example in case of recurring epidemic, elements of the system can be stored in the manufacturing sites. As the manufacture from pre-manufactured elements of the system is fast and simple, it can be made even without extensive expert knowledge according to the need that arises.

The pathogenic agent can be either a cellular microorganism or a virus.

Similarly, in case of cancer, capturing molecules against CTCs can be stored and devices of the invention can be manufactured and applied very quickly.

In any case the versatility of the method can also be utilized in case of personalized medicine. Once diagnosis of a subject results in the identification of a given pathogen, if an appropriate capturing molecule exists against said pathogen, the device of the invention can be prepared from the pre-made elements very fast and treatment with blood cleansing started.

The present invention can be particularly well utilized in cases of endemics due to war. The most frequent pathogens which typically arise in conditions of war can be categorized into a few dozens. Thus, it is not impossible to prepare with capturing molecules

Also in case of poisoning a device of the invention can be quickly tailored according to need. Most typical protein type food toxins are known an capturing molecules or antitoxins are also known against such food toxins. Thus, a device against typical toxins can easily be manufactured according to the need as it arises.

The system or kit comprises elements as described herein.

The diameter of the beads provide an sufficient surface volume ratio with sufficient capturing sites and with a space to allow sufficient flow velocity allowing laminar flow.

Said fillable cartridge is useful for making blood flowing through said cartridge. Said fillable cartridge has a blood inlet and a blood outlet and has an outlet diaphragm and an inlet diaphragm to withhold the beads.

Preferably the beads are non-porous beads.

Preferably the cartridge comprises multiple types of beads i.e. beads with multiple capturing molecules against multiple pathogenic materials (antigens).

Preferably, the cartridge having a volume of its useful space (i.e., wherein the beads are placed) of 20 to 500 ml, preferably 100 to 500 ml, preferably 300 to 400 ml in case of an adult human subject, preferably an adult human subject.

In a preferred embodiment the useful space is at least 20 ml in case of a human subject, preferably an adult human subject. An adult human has about 4.5 to 5.5 litres of blood.

Thus, the volume of the cartridge is at least 4 ml per liter of total blood volume of the subject. It can be calculated for the lower limit of the range of a subject.

However, preferably the volume of the cartridge and the space filled in by the beads is higher to provide a safely sufficient amount of capturing sites to arrive at a quick blood cleaning.

However, it shall be considered that an amount of blood filling this space of the cartridge is lost and is therefore to be minimized. Therefore the volume of the cartridge (i.e. a volume of its useful space, i.e., wherein the beads are placed) is, in case of an adult human subject is 100 to 500 ml, preferably 300 to 400 ml.

A mesh as used herein is a separating wall with pores having a pore size smaller than the lowest diameter of the beads so a to withhold the entirety of the beads within the cartridge. Preferably both the first (inlet) mesh and a second (outlet) mesh are applied. Preferably both meshes have the same properties and thus the direction of the flow is reversible.

The pore size of the mesh is smaller than the diameter of the beads, or if there are beads with multiple diameters smaller than the smallest diameter to withhold the beads.

The mesh that withholds the beads may be e.g. of polyester or polypropylene.

Preferably, the system or kit also comprises tubing for making blood flowing through.

Preferably, the system or kit also comprises derivatization agents, for example as described in the examples or herein. Such methods and agents are known in the art.

Preferably the surface of said beads is or can be covered by a substance reducing non-specific capturing of the pathogens. In a preferred embodiment such non-specific binding reducer also forms part of the system or kit.

Preferably, the system or kit according to the invention is useful to prepare a device wherein the number of the specific binding sites is at least 50,000 per ml, preferably at least 70,000 per ml or at least preferably 100,000, in highly preferred embodiments at least 0.5×10⁶ or at least 1.0×10⁶ or at least 1.5×10⁶.

Highly preferably, the system or kit according to the invention is useful to prepare a device wherein the specific binding sites is at least 30,000 per ml, preferably at least 40,000 per ml or at least preferably 50,000 per ml.

In particular the cartridge is useful to specifically capture at least 30,000 cells per ml, preferably at least 40,000 cells per ml or at least preferably 50,000 cells per ml.

In case of viruses and toxins a much higher number of binding sites can be made. However, this may be necessary as such pathogenic agent are often present in a higher number in the blood. Thus, it is contemplated that the same bead diameter range and other parameters are applicable for such pathogenic material as well.

A cartridge of the invention has a useful volume (i.e. a volume of the suspension of beads of 300 to 500 ml which means that the device is capable of capturing (has a capacity of), in case a relatively smaller cartridge, at least 0.9×10⁶ binding sites, preferably at least 1.2×10⁶ binding sites, more preferably at least 1.5×10⁶ binding sites, or in case a relatively larger cartridge (e.g. of 500 ml), at least 1.5×10⁶ binding sites, preferably at least 2×10⁶ binding sites, more preferably at least 2.5×10⁶ binding sites.

For example, in case of circulating cancer cells (CTCs) an average cancer patient's 7.5 ml blood contains ˜5 cells in case of a poor prognosis, which is significantly lower than the capturing ability of the anti-EpCAM immobilized beads provided in preferred embodiments of the invention.

The flow rate may be calculated per ml of the device cartridge as about 2 ml/min per cm³ of the cartridge volume or in a broader range a 1 to 3 2 ml/min per cm³ or 1.5 to 2.5 ml/min per cm³. Thus, in case of an about 2.5 cm³ cartridge (i.e. a cartridge containing about 2.5 cm³ beads of 400 μm (or diameter of 350 μm to 500 μm, to give a broader range) an advisable volume flow rate is ab 2.5 to 7.5 ml/min, or preferably 3.75 to 6.25 ml/min, in particular about 5 ml/min.

In case of a 300 ml cartridge the advisable flow rate is in the range of 300 to 900 ml/min, or 400 to 700 ml/min or about 500 or 600 ml/min.

This will provide a robust approach which is able to capture pathogenic material from blood in a broad range of application.

Based on these results, the inventors propose the use of beads with a diameter of 300 μm or 350 μm to 500 μm, preferably 350 to 450 μm, highly preferably 400 to 450 μm and variants of these limits. In a highly preferred embodiment cartridges with about 400 μm diameter beads are used.

The size range expressed in diameters of the beads is important because if the diameter is too low then shear forces increase and throughput (the volume of the blood flowing through during unit time) is decreased. On the other side if the diameter of the beads is too large then the surface per volume ratio is decrease and the void volume of the system is increased which increases the loss of the amount of blood of the patient.

The inventors have found that using the diameter range given herein all these requirements are met at the same time. This is important in the present invention as the present system is robust and versatile to cover various pathogens with the same platform. As found herein, the system is sufficiently robust to be able to capture a wide range of pathogens like viruses, cells or toxins, in particular viruses or cells.

The material of the device is a biocompatible material, preferably a biocompatible plastic e.g. polycarbonate . . . .

The sealing may be made of e.g. silicone or other inert and resilient material.

The mesh that withholds the beads may be e.g. of polyester or polypropylene.

The material of the beads may be any plastic material which can be derivatized to capture a protein type capturing molecule, said plastic material including but not limited to polypropylene, PET, polycarbonate, PMMA, PDMS, HD/LD-PE. In alternative embodiment the beads can be prepared of e.g. polystyrene, polysulfone, etc.

The bead also can be made of silica.

“Cartridge” as used herein is small container, as provided herein, that is hollow and can be filled with material, e.g. beads as explained herein, and which can be used as a part of a device or a system, e.g. a blood cleansing system as provided herein.

A “bead” is a small rounded object or article made of solid material. A “rounded” i.e. one lacking sharp angles and/or having gentle curves. In a particular embodiment the bead is spherical.

In an embodiment the capturing molecule is selected from a group consisting of proteins with specific binding site(s), in particular antibodies, glycoproteins, in particular mucins and/or lectins, oligonucleotide capturing agents like aptamers, small capturing molecules and ligands, in particular folic acid and any combinations of thereof.

In a preferred embodiment the capturing molecule is a protein-type capturing molecule e.g. an antibody, a microbody, a one-domain antibody etc.

In a particular embodiment the capturing agent is an antibody or a binding fragment thereof or a biomolecule having a binding region of an antibody, said antibody being preferably a tumor specific antibody, in particular an antibody adapted to said mammal, more preferably an antibody selected from the group of anti-CD44 and anti-EpCAM antibodies.

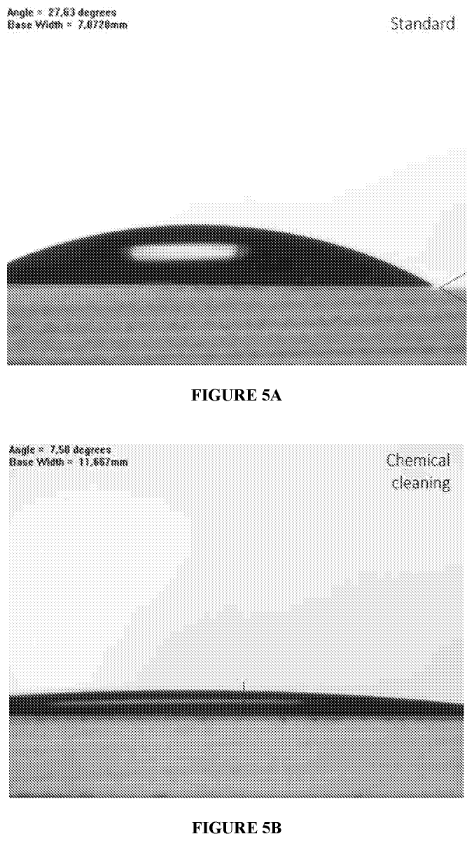

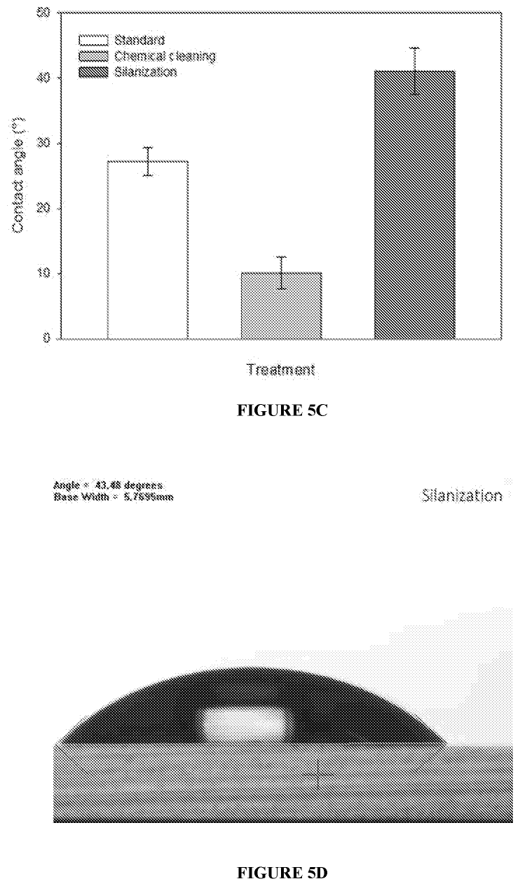

As to linear flow velocities it has been found by the present inventors that a higher linear flow rate results in lower non-specific binding whereas specific binding remains at a high level. Thus, the method is more selective with relatively high linear flow rates (see FIG. 5a).

Further parameters to be considered is the total time of the treatment and shear rates. An overly high shear rate is no disadvantageous from multiple aspects.

Blood components may be sensitive for high shear forces.

Moreover, with the present method clusters of pathogenic agents, if present, also can be captured. For example, CTCs are ready to form such clusters binding of which may be advantageous.

It is preferred if the parameters of the device allow cell-to-cell binding or binding of clusters to the beads.

In a preferred embodiment the linear flow rate is preferably between 60 to 600 cm/min, preferably 60 to 300 cm/min, highly preferably 60 to 120 cm/min (equal to 1-10 cm/s, 1-5 cm/s 1-2 cm/s i.e. 0.01 to 0.1 m/s or 0.01 to 0.05 m/s or 0.01 to 0.02 m/s respectively) which provides an effective capturing still a low shear rate.

Taking into account the linear flow rate is preferably between 50 to 100 cm/min which provides an effective capturing still a low shear rate.

According to an embodiment of the invention the system comprises the kit (of parts) as defined above as well as means to operate the device. For example the system may comprise a pump to pass the blood of the subject through the device wherein the flow velocity can be set and regulated as describe above.

Moreover the system may comprise a control unit able to operate the pump and thereby regulate flow velocity.

In a preferred embodiment the pump can be operated from a microprocessor or a computer connected or being part of the control unit and programmed to set the flow rate and maintain it within limits.

In a further embodiment the system comprises means to measure certain parameters of the effluent, cleansed blood, for example the level of the pathogenic material can be monitored.

In an embodiment the data from such monitoring are supplied into the control unit having microprocessor or into the computer and used as input data or feed-back in the program. For example a threshold level for the level of the pathogenic material can be defined and once the level of the pathogenic material is below that threshold the blood cleansing process may be stopped.

Further, if the level of the pathogenic material is above threshold, however, does not lowered in time or between to measurement in the monitoring a signal may be generated suggesting that there is an error in the operation of the device.

The program may also receive input signals on the status of the patient, for example status of heart, blood pressure, etc. Again, if those parameters a within pre-defined limits the process can be continued. However, if any of the parameters are within a range which indicates a problem in the status of the patient a signal can be generated to warn the operator or medical personnel or the doctor. In certain cases or when any of the parameters is in a predefined zone out of normal range the method can be stopped by the control unit of the system.

A laboratory scale capture device was designed and fabricated to evaluate the feasibility of a novel immune-affinity capture based extracorporeal treatment approach.

The invention also relates to the device manufactured and comprising the cartridge and the derivatized beads comprising the capturing molecules on their surface.

The invention also relates to a method of manufacture of said device.

In an embodiment the derivatized beads are obtained by modification of glass beads.

In a further embodiment the derivatized beads are obtained by modification of plastic (??) beads.

Both the glass beads and the plastic beads can be commercially available beads. Exemplary methods for derivatization and providing beads to carry a large number of capturing molecules are shown for both types of beads.

While the possibility of utilization of cheap commercially available beads is an advantage of the present invention, and the examples shown herein have a particular utility, the method can be adapted to any types of beads of the claimed size range and having an appropriate density of capturing molecules.

A laboratory scale hemoperfusion system was designed and fabricated to study the feasibility of the proposed approach, which combines the specificity of affinity chromatography with high throughput of hemoperfusion technique.

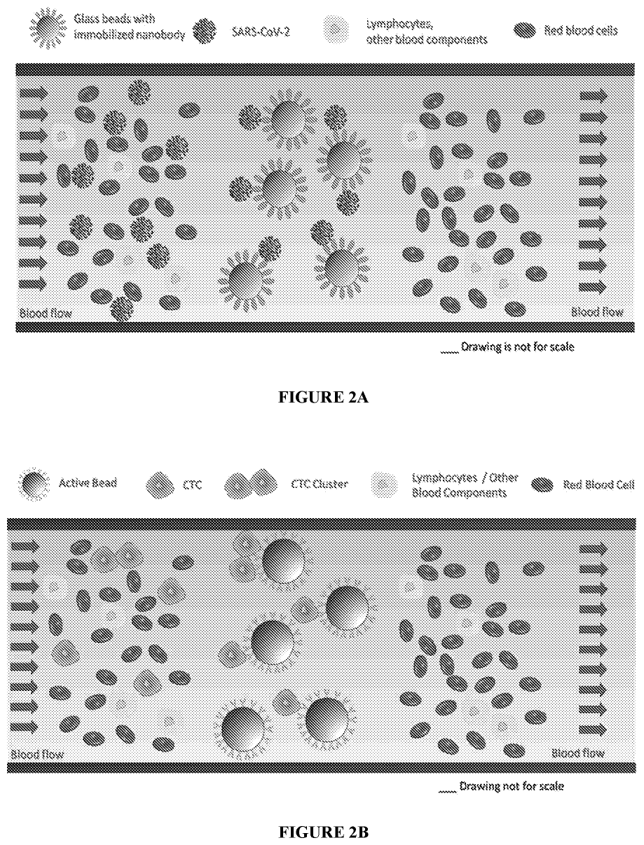

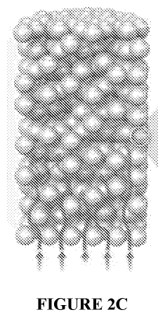

The scheme of the proposed capture technology is shown in FIG. 2a for SARS-Cov2 (as example for viruses) and FIG. 2b for CTCs (as example for cells) showing a schematic representation of the capture mechanism.

In an example, the down-scaled laboratory model device filled with 2.6 cm³ of glass beads captured 120,000 virus particles from virus culture media circulation, suggesting the partitioning ability of 15 million virus particles with an actual therapeutic size column. In other words, since the actual-size of an average hemofiltration cartridge is about 330 cm³, the virus-binding (virus-capturing) capacity of our design is expected to be around 15 million. There is a reported correlation between human plasma SARS-COV-2 virus concentration and COVID19 disease course and mortality [Fajnzylber, J., et al. 2020] [24, 47]. Viremia is observed in 27% of hospitalized COVID19 patients and typical virus concentrations in the plasma of viremic patients range from 100 to 1000 copies/ml [Colagrossi, L., et al. 2021] [48]. Considering that an adult has an average of five liters of blood, the amount of SARS-COV-2 to be captured from the bloodstream at one time is expected to be around 5 million virus particles, so the system design reported in this paper holds the promise to have three-fold excess capture capacity. Please note that the reported virus copy numbers in the literature represent the genomic concentrations i.e., albeit the exact relationship between genomic virus concentration and infectious virial load level is still under debate [Wrapp, D., et al. 2020; van Doremalen, N., et al. 2021; Israelow, B., et al. 2021] [41, 49, 50], all reported findings in this study should be interpreted from the technical feasibility point of view only. However, the actual therapeutic benefit of the proposed approach cannot be evaluated without comprehensive clinical trials. After further development the reported technology could serve as the base of an artificial organ platform, which represent an option to prevent organ failure and improve survival.

In a preferred embodiment the blood purification device offers efficient capture and removal of the targeted virus from human cardiovascular circulation, thus decreasing virus load.

In an example, single domain antibodies against the VHH-72 virus variant produced by recombinant DNA technology were immobilized on the surface of glass micro-beads, which were then utilized as stationary phase. The virus suspension was flown through the immune-affinity device that captured the viruses and the filtered media left the column. The feasibility test of the proposed technology was performed in a BSL-4 classified laboratory using the actual SARS-COV-2 strain. The laboratory scale device specifically captured 120,000 virus particles from the culture media circulation, which corresponded to 15 million virus particles capture ability of a potential therapeutic column. This represents three times over-engineering with the assumption of 5 million genomic virus copies in an average viremic patient. Our feasibility results suggested that this new therapeutic virus capture device could significantly lower virus load thus prevent the development of more severe COVID-19 cases and consequently reducing mortality rate.

In an embodiment the setup has been tested in a BSL4 laboratory environment with a live virus suspension as representative conditions to explore any potential risk factors before further development. In a highly preferred embodiment the feasibility of the system has been discovered by removing SARS-COV-2 from patient's blood. Thus, the present invention is also useful to fight the corona virus pandemic.

The setup has been tested in a BSL4 laboratory environment with a live virus suspension as representative conditions to explore any potential risk factors before further development. Capture efficiency was investigated using an actual strain of SARS-COV-2. In the presented proof of concept experiments, the virus particles were captured from a culture medium as described in Example 2 as a fluidic circulation model in a model experiment. Virus concentrations were determined by ddPCR, both in the active and control experiments (Table 1).

As shown in this experiment the surface-to-volume ratio of this laboratory scale experimental setup was significantly higher than that of in an actual hemoperfusion device, thus non-specific binding was overrepresented. During the two-hour experiment, the active cartridge captured 32% of the virus particles from the circulation, while the control cartridge was able to remove only 7%, apparently representing non-specific binding.

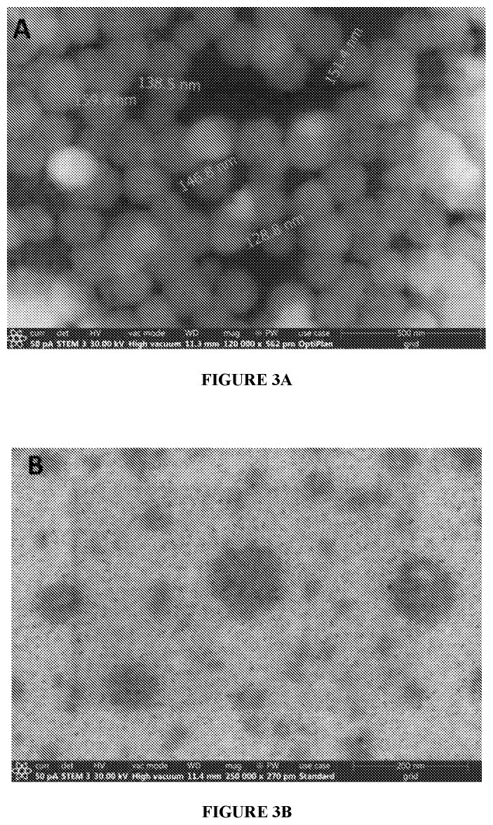

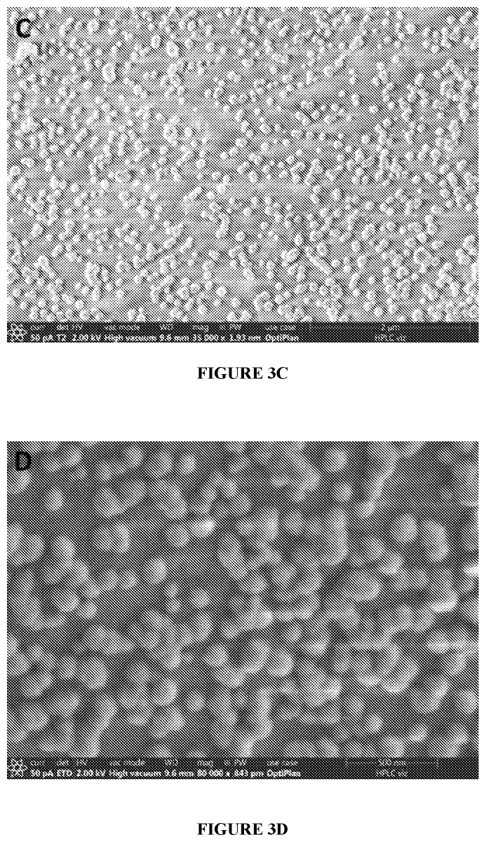

After the capture efficiency studies the captured viruses (images of virions) on the surface of the beads have been shown by scanning electron microscope (SEM) imaging was utilized. FIGS. 3.A and 3.B. FIGS. 3.C and 3.D show the captured virus particles (virions) on the surface of the beads.

Specific capture from buffer model solution was determined by flow cytometry. The scheme of the proposed technology is shown in FIG. 2.b

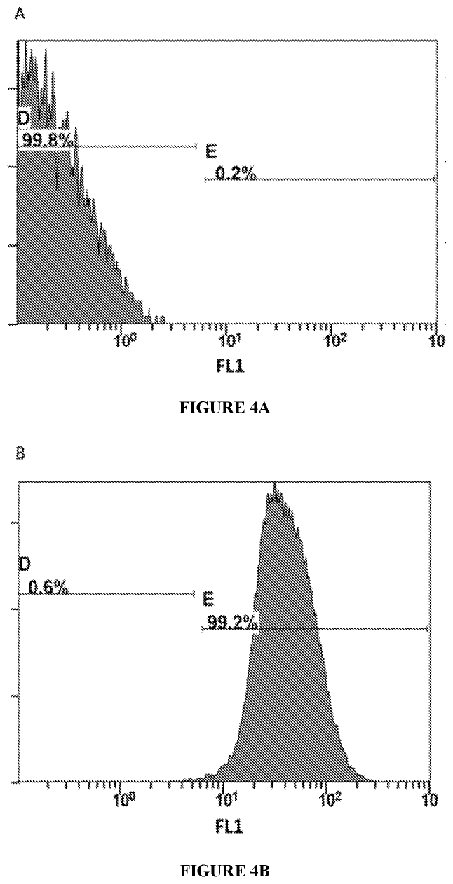

Goniometry was used to examine the quality of the prepared glass surfaces. Glass slides were chemically cleaned and silanized as described above. Efficiency of each step were examined after drying under laminar hood. Evaluation of contact angles was carried out by FTA32 software (FIG. 4). After chemical cleaning, the number of hydroxyl-groups on the surface of the glass increases raising hydrophilicity, resulting in the decrease of the contact angle significantly compared to the initial state. APTES molecules bind to these hydroxyl groups, increasing the hydrophobicity of the surface, which causes an increase in the contact angle.

A “capturing molecule” is a molecule that specifically binds to the target molecule with a desired binding affinity, i.e. with a binding affinity sufficient to form a complex between the binding molecule and the target molecule. Preferably the binding molecule is a monospecific binding agent capable of binding to a single epitope or antigenic protein. Thus, preferably, capturing and binding may by uses as synonyms in terms of binding whereas capturing has the context of binding a molecule to remove it from a medium. In an embodiment the binding molecule is an antibody or a fragment thereof; in that case the monospecific binding agent will therefore be a monoclonal antibody or a fragment thereof, which can be obtained from a hybridoma or expressed from a cloned coding sequence. An example of a suitable antibody fragment is a part of an antibody that comprises an antibody binding part comprising a complementarity determining region (CDR). Such binding parts can be inserted into a natural or synthetic scaffold molecule. A capturing molecule can be derived from a naturally occurring molecule, e.g. from an antibody. Thus, capturing molecules include proteins of antibody derived protein scaffolds, like fragments, single-domain antibodies, single chain antibody fragments (scFv), Fab fragments nanobodies [Muyldermans S. “A guide to: generation and design of nanobodies” The FEBS Journal (2021) 288 2084-2102].

Alternatively, a capturing molecule can be entirely artificially made, e.g. as a synthetic peptide. Thus, capturing molecules include proteins of non-antibody protein scaffolds like fibronectin, lipocalins, anticalins, affibodies, etc. [Stern et al. “Alternative non-antibody protein scaffolds for molecular imaging of cancer” Current Opinion in Chemical Engineering (2013) 2 425-432, Škrlec K. et al. “Non-immunoglobulin scaffolds: a focus on their targets” Trends in Biotechnology, (2015), 33(7) 408-418; Gebaure and Skerra “Engineering of binding functions into proteins” Current Opinion in Biotechnology (2019), 60 230-241].

“Blood” means the a body fluid in the circulatory system of an animal (animal blood), that carries and delivers substances including nutrients and oxygen to the cells and transports metabolic waste products away from the cells of the animal, wherein the animal is a vertebrate, preferably avian or mammalian, more preferably mammalian, in particular human blood, blood components, and products made from blood.

“Pathogenic material” is understood herein as biological material harmful to a subject's health i.e. which is able to result in a disease (harmful biological material). Thus, in this sense pathogenic material includes harmful i.e. pathogenic cells including circulating neoplastic cells or tumor cells as well as pathogenic microorganisms like bacteria, fungi and antigenic toxins, which can be bound to a surface via a capturing molecule, typically a protein capturing molecule. In an embodiment the pathogenic material is a bloodborne pathogen, i.e. a pathogen carried by the blood of a patient, preferably an infectious pathogen, in particular a pathogenic microorganisms that is present in animal blood and can cause disease in the animal as defined herein, preferably in a human. In a particular embodiment the bloodborne pathogen is one defined by US Occupational Safety and Health Standard No. 1910.1030, Appendix A.

A “patient” is a subject who is under medical treatment or care or surveillance or inspection or diagnosis. The subject is an animal having blood as defined herein.

In this study we proved the feasibility of immobilization of human anti-EpCAM antibodies onto the surface of glass beads for selective capture of circulating tumor cells from the bloodstream. The capture ability of modified glass beads in the laboratory scale system can reach over 130,000 cells next to higher flow rates and based on the results of the present study, the capture ability could even reach 16.5 million cells/cartridge in case of a clinically applicable size device, which is significantly higher than required in case of clinical applications. Anti-EpCAM could be replaced by any bioactive molecule that could be immobilized, like other specific antibodies, nanobodies, nucleotides, peptides or the combination of those, depending on the application of the cartridge. Since cells can be mobilized from the surface of the beads using trypsin-EDTA, which is also used during cell culture, they are suitable for further molecular or pathology studies and diagnostic purposes.

In an important alternative embodiment plastic beads of the same size range are applied.

Biomolecules are commonly immobilized onto solid surfaces for clinical applications and polymers are frequently used as support materials next to silicone (PDMS) and glass surfaces [Barbosa, A and Reis, N. 2017] [1]. Creation of polar surface function groups through plasma functionalization [Sathish, S. et al. 2019; Járvás G. et al., 2018] [2, 3] or chemical cleaning [Wardani, A. K., et al. 2019] [4], amination of surfaces and cross-linkers allow covalent immobilization methods [Fixe, F., et al. 2004] [5], In a preferred example poly(methyl-methacrylate) (PMMA) surface glass beads are applied. The main disadvantage of polymers is their low surface free energy, causing reduced hydrophilicity and adhesion. These properties can be significantly changed by different methods for polymer surface alterations, such as wet chemical cleaning treatments [Wardani, A. K., et al. 2019; Cheng, J. Y., et al. 2004] [4, 8], UV-ozone exposure, laser alteration and plasma treatments [Rezaei, F., B. et al. 2016] [9]. Particularly, PMMA surfaces are covered by stable ester-methyl groups, decreasing the efficacy of the standard chemical modification techniques [Acsente, T., et al. 2016] [10]. In this case, we applied a simple wet chemistry method, removing any surface contamination with abs. ethanol/isopropanol and oxidation of methyl-ester groups with 20% sulfuric acid.

The accessible methyl-esters of the native poly(methyl-methacrylate) (PMMA) react with hexamethylenediamine, yielding primary amines on the surface, under basic pH conditions. Amino-groups of the surface and proteins are cross-linked by glutaraldehyde [Fixe, F., et al. 2004] [5, 7].

3-Aminopropyltriethoxysilane (APTES) can be used for PMMA surface amination after sulfuric acid or other oxidizing treatments. APTES can be dissolved in dry ethanol [Járvás, G., et al. 2018] [3], water [Vakili, M., et al. 2019] or acetonitrile [Miranda, A. et al. 2020] [12]. Amino-groups of the silanized surface and proteins can be cross-linked by glutaraldehyde [Járvás, G., et al. 2018] [3].

### PARTICULAR EMBODIMENTS

### Blood Purification to Remove Pathogenic Viruses

Inspired by various hemoperfusion and immune-affinity systems, a blood purification device has been developed that offers highly efficient capture and removal of the targeted virus from human cardiovascular circulation, thus decreasing virus load. Single domain antibodies against the VHH-72 virus variant produced by recombinant DNA technology were immobilized on the surface of glass micro-beads, which were then utilized as stationary phase. The virus suspension was flown through the immune-affinity device that captured the viruses and the filtered media left the column. The feasibility test of the proposed technology was performed in a BSL-4 classified laboratory using the actual SARS-COV-2 strain. The laboratory scale device specifically captured 120,000 virus particles from the culture media circulation, which corresponded to 15 million virus particles capture ability of a potential therapeutic column. This represents three times over-engineering with the assumption of 5 million genomic virus copies in an average viremic patient. The present feasibility results have shown that this new therapeutic virus capture device could significantly lower virus load thus prevent the development of more severe COVID-19 cases and consequently reducing mortality rate.

### COVID-19—Background for an Example for Viral Pandemic and its Significance.

Since the first hospitalizations in December 2019 [Wu, F., et al. 2020] [1], and the detection of the novel coronavirus in January 2020 [Zhou, P., et al. 2020] [2], the COVID-19 outbreak causes public healthcare and social crisis worldwide [Haldane, V., et al. 2021] [3], and apparently remains for years [Kissler, S. M. and C. Tedijanto 2020] [4]. During the first year of this pandemic, almost 100 million people got infected [5]. PCR methods and lateral flow tests are the most widely used diagnostic tools [Weissleder, R., et al. 2020; Vandenberg, O., et al. 2021] [6, 7], but CRISPR/CAS systems [Rahimi, H., et al. 2021] [8], antibody functionalized bioactive nanomaterials and other technologies were also implemented to overcome this unseen global challenge [Guo, K., et al. 2021; Tang, Z., et al. 2020; Sheridan, C. 2020] [9-11]. Parallel to the diagnostic endeavors, new therapy strategies have been utilized and/or developed, such as antivirals, convalescent plasma treatments, antibody therapies, extracorporeal treatments and blood purification methods for clinical use. Furthermore, vaccine development against severe acute respiratory syndrome coronavirus 2 (SARS-COV-2) was immensely accelerated [Mullard, A. 2020] and as of the beginning of 2022, vaccination seems to be an effective way of immunization [Swol, J. and R. Lorusso 2020; Subbarao, K. 2020; Graham, B. S. 2020] [13-18]. However, naturally occurring genetic viral mutations resulted in different variants prolonging the challenge [Tao, K., et al. 2021] [18].

Approximately 17-30% of SARS-COV-2 positive patients reported no typical symptoms [Rasmussen, A. L. and S. V. Popescu; 2021] while on the other hand, about 15-30% of patients develop respiratory failure requiring hospital admission, 12% needs ventilation and 3% extracorporeal life support [Xi, Y. 2020] [14]. Cardiovascular complications including thrombosis [Varga, Z., et al. 2020] and systemic, cytokine-mediated inflammation are also associated with severe SARS-COV-2 infection. Most importantly, high viral load (i.e., viremia) predicts poor surviving prognosis [Fajnzylber, J., et al. 2020; Pujadas, E., et al. 2020; Tsukagoshi, H., et al. 2021] [24-26]. Later in the disease course, approximately two weeks after the onset of the symptoms and viral replication in the upper respiratory tract, spreading starts in the lower respiratory tract and creates secondary viremia followed by an attack against ACE2 expressing organs [Cao, W. and T. Li 2020] [27]. This progress of spreading correlates with clinical deterioration, which usually occurring roughly two weeks after the onset of the symptoms. In addition, other organs possibly reached directly by the virus through the circulation during persisting or temporary viremia may also occur in case of severe COVID-19 cases [Müller, J. A., et al. 2021; Perotti, C. and C. Del Fante, 2020; Xu, Z., et al. 2020] [28-30]. High plasma viral loads also indicate increased severity, potential organ damage and high chance of mortality [Fajnzylber, J., et al. 2020; Cao, W. and T. Li 2020; Hagman, K., et al. 2020; Eberhardt, K. A., et al. 2020; Xu, D., et al. 2021] [24, 27, 31-33].

Extracorporeal membrane oxygenation and extracorporeal CO₂ removal can be used to address respiratory failures. Furthermore, cytokine removal by continuous renal replacement therapy or direct hemoperfusion with filters, HA resin hemoperfusion cartridges and Toraymixin polymixin-B endotoxin removal are available therapeutic strategies in case of cytokine storm cause by SARS-CoV-2 infection [Swol, J. and R. Lorusso 2020; Barbaro, R. P., et al 2021; Ronco, C., et al. 2021] [13, 34, 35]. Early cytokine removal may prevent the progression of respiratory failure or other organ dysfunctions in critically ill patients [Ramírez-Guerrero, G., et al. 2021; Shadvar, K., et al. 2021] [36, 37].

Next to the hemofilters, hemoperfusion and plasmapheresis procedures are used to non-specific pathogen removal from the bloodstream such as heparin immobilized polyethylene beads. The end-point attached heparin binds to viruses, bacteria, fungi and toxins similarly to heparane-sulfate interacting with cell-surface [Seffer, M. T., et al. 2021; Pape, A., et al. 2021] [38, 39]. Hemopurifirer lectin affinity plasmapheresis filters have also been designed for whole virus, exosome and exosomal microRNA removal and investigated for COVID-19 therapy [Amundson, D. E., et al. 2021] [40].

### Blood Purification to Remove Circulating Cancer Cells

The majority of cancer deaths are due to metastases, in which circulating tumor cells (CTCs) detaching from the primary tumor play an important role. To prevent further progression of the disease, metastases should be stopped from developing. Hereby, we report that EpCAM-positive CTCs can be selectively captured by anti-EpCAM antibodies covalently immobilized on the surface of beads of around 400 micro, which can be, after scale-up, an alternative to existing cancer therapies. The essential steps are described herein. The evaluation of the immobilization process, capturing efficiency, and the effect of linear velocity and anticoagulants on specific capture were demonstrated by spiking HTC116 cells into phosphate buffered saline and ran through a laboratory scale test system, including model circulation system, representing hemoperfusion with a cartridge, filled with the activated beads. The captured cells were quantitatively measured by flow cytometry.

### CTCs—Background for an Example for Cancer Treatment and its Significance

Circulating tumor cells (CTC) were first reported more than 150 years ago by Thomas Ashworth, who found malignant cells in the bloodstream during an autopsy [Ashworth, T. R. et al. 1869] [1]. Besides that, CTCs have great prognostic value [Brisotto, G., et al. 2020] [2] carrying important information about tumor heterogeneity and genetic mutations [Wu, M., et al. 2018] [3], and CTC related discoveries are a big step forward to personalized medicine and therapy management, metastases caused by circulating cells detaching from the primary tumor are the leading cause of death in cancer patients [Schochter, F., et al. 2019; Tayoun, T., et al. 2019; Keup, C., et al. 2020; Cimadamore, A., et al. 2020] [4-7]. The exact mechanism of the metastatic process still has not been completely understood at the molecular level [Mamdouhi, T., et al. 2019] [8]. Exosomes might play a major role in the forming of metastases, as they transmit signals that regulate cellular behavior, EMT (epithelial to mesenchymal transition) and organotropism [Chicon-Bosch, M. et al. 2020; Yang, B., et al. 2020] [9, 10].

Increased sensitivity of detection methods, depending on both immuno-capture and label-free CTC enrichment or ctDNA analysis, allows liquid biopsy, which is still relatively new to clinical practice and has its limitations [Ignatiadis, M. 2021; Rushton, A. J., et al. 2021] [11-14]. Nonetheless, these methods are exciting innovations to identify biomarkers at different stages of the disease course in a non-invasive way [Brisotto, G., et al. 2020; Cimadamore, A., et al. 2020] [2, 7]. Rarity and heterogeneity are key issues of these real-time liquid biopsy technologies [15]. Since 1-10 CTCs are present per a billion of blood cells in the circulation [16, 17] it is not realistic that only 7.5 ml of whole blood is used for liquid biopsies performed by microfluidic systems (CellSearch; ClearCell FX) [18, 19]. Furthermore, existing technologies are more-likely designed for capturing single CTCs, not CTC clusters, which also may be present in the circulation, [20, 21].

By removing CTCs from the bloodstream, development of metastases could be prevented. These methods may be based on technologies such as immuno-capture with specific antibodies covalently bound to the surface of the device [Gaitas, A. and G. Kim 2015] [22], non-specific capture [Kang, J. H., et al. 2014] [23], or photo-immunotherapy [Kim, G. and A. Gaitas, 2015]. Extracorporeal photopheresis (ECP) can be applied in case of leukemia and T-cell lymphoma [Garban, F., et al. 2012; Vieyra-Garcia, P. A. and P. Wolf 2020] [25, 26]. During ECP treatment, the leukocytes are exposed to 8-methoxypsoralen photosensitizer and ultraviolet-A light. Nonetheless, the existing procedure has limited selectivity and efficiency and produces partial response in the majority of treated patients, expensive and takes a very long time [Darvekar, S., et al. 2020] [27]. Another interesting modality has been proposed by Gaitas and Kim [Gaitas, A. and G. Kim 2015] [22]. They examined the cell-capture efficiency of the immune-activated inner wall of a PDMS polymer tube. This technic also has been investigated for not just CTCs, but bacteria removal [Kim, G., H. Vinerean, and A. Gaitas, 2017] [28]. Modification of hemodialysis membranes could also be an option for high volume CTC removal therapy [Jarvas, G., et al. 2021] [29].

### EXAMPLES

### Example 1—Materials and Methods

### Chemicals

Picoline borane, DMEM cell culture media and phosphate buffered saline (PBS, pH 7.4) were from Sigma-Aldrich (St Louis, MO). Polyethylene glycol was purchased form Merck (Kenilworth, NJ). Fetal bovine serum, trypsin-EDTA and 3-aminopropyltriethoxysilane (APTES), calcein-AM, fluorescein isothiocyanate (FITC) labelled anti-EpCAM, anti-EpCAM, trypsin-EDTA and penicillin-streptomycin were from Thermo Fisher Scientific (Waltham, MA). Glutaraldehyde was purchased from Carl Roth Chemicals (Karlsruhe, Germany). Methanol and absolute ethanol were purchased from VWR (Radnor, PA). Hydrochloric acid, sulfuric acid and dry toluene and glutaraldehyde were purchased from Molar Chemicals Kft. (Halasztelek, Hungary). Kanamycin was from SERVA Electrophoresis GmbH (Heidelberg, Germany). Isopropyl β-D-1-thiogalactopyranoside (IPTG) was from Biosynth AG (Staad, Switzerland). The SDS PAGE gel was made from 40% acrylamide solution from Bio-Rad (Hercules, CA) and HPLC grade water in a ratio of 37.5:1. Buffer ‘A’ contained NaH₂PO₄ from Merck (Kenilworth, NJ) and NaCl from VWR (Radnor, PA). Imidazole for buffer ‘B’ was purchased from Merck. Polyethylene-glycol was obtained from Merck (Kenilworth, NJ). FlowCount Fluorospheres were purchased from Beckman Coulter (Brea, CA). Heparibene Na 25000 IU solution for injection was from TEVA (Debrecen, Hungary).

### Expression and Purification of SARS-COV-2 Spike Protein Specific Single Domain Antibody

The coding sequence of the publicly available single domain antibody (sdAb) against SARS-CoV-2 spike protein was codon optimized for E. coli [Wrapp, D., et al. 2020] [41]:

(SEQ ID NO: 1)
(CAGGTGCAGCTGCAGGAAAGCGGCGGCGGCCTGGTGCAGGCGGG
CGGCAGCCTGCGCCTGAGCTGCGCGGCGAGCGGCCGCACCTTTAG
CGAATATGCGATGGGCTGGTTTCGCCAGGCGCCGGGCAAAGAACG
CGAATTTGTGGCGACCATTAGCTGGAGCGGCGGCAGCACCTATTA
TACCGATAGCGTGAAAGGCCGCTTTACCATTAGCCGCGATAACGC
GAAAAACACCGTGTATCTGCAGATGAACAGCCTGAAACCGGATGA
TACCGCGGTGTATTATTGCGCGGCGGCGGGCCTGGGCACCGTGGT
GAGCGAATGGGATTATGATTATTATCTGGATTATTGGGGCCAGGG
CACCCAGGTGACCGTGAGCAGC)

The genes with 5′-Nde1 and Xho1 cleavage sites were synthetized by Twist Bioscience (South San Francisco, CA) and cloned into a pET28b expression vector (Novagen, Darmstadt, Germany). Shuffle T7 Express E. coli (New England Biolabs, Ipswich, MA) cells were transformed with the plasmids, according to the supplier's instructions. 5 mL LB pre-culture supplemented with 50 μg/mL kanamycin (Kan) was prepared as reported earlier [Reider, B., et al. 2021; Meszaros, B., et al. 2020] [42, 43], and the resuspended cells were used to inoculate 1 L LB/Kan. The cell culture was grown at 30° C. with 140 rpm shaking in a baffled flask until the OD₆₀₀ value reached the 0.6-0.8 range. Protein expression was induced by the addition of 0.4 mM isopropyl β-D-1-thiogalactopyranoside (IPTG) and incubated overnight. The cells were harvested by centrifugation at 6000×g for 30 min, then washed with buffer ‘A’ (20 mM NaH₂PO₄, 500 mM NaCl, pH 7.5). Cells were resuspended on ice in 10 ml buffer ‘A’ containing EDTA-free Mini Complete protease inhibitor (Roche, Basel, Switzerland) and disrupted by sonication (8×30 s, 40% amplitude, Omni Sonic Ruptor, Perkin Elmer, Waltham, MA). After centrifugation and filtration, the supernatant was loaded onto a Ni (II)-saturated, pre-equilibrated 5 ml HiTrap Chelating column (GE Healthcare, Chicago, IL) and purified using a linear gradient of 25-300 mM imidazole containing buffer (5-60% ‘B’ buffer, 11 column volume). The pure protein was eluted at 250 mM imidazole concentration and dialyzed. Purity of sdAb was confirmed on 15% SDS PAGE gels and protein concentration was calculated using the following parameters given by ProtParam [Reider, B., et al. 2021] [42]: aSARS-6His, 16.2 kDa, 37,025 cm⁻¹M⁻¹. The application of the optimized production protocol resulted in a typical yield of pure anti-SARS of 5 mg/L culture.

Immobilization of Anti-SARS-COV-2 sdAb onto the Surface of Glass Beads

Commercially available monodisperse 450 μm diameter soda-lime glass beads were purchased from Glass Sphere (Jablonec nad Nisou, Czech Republic). The SARS-COV-2 spike protein specific sdAb was immobilized onto the surface of the beads as follows. First, the beads were treated with a 1:1 mixture of cc. hydrochloric acid and methanol for 30 minutes at room temperature, followed by washing with HPLC grade water. After the washing step, the beads were treated with cc. sulfuric acid for 30 minutes at room temperature. The beads were washed and treated by boiling HPLC grade water for 30 minutes [Cras, J. J., et al. 1999] and dried in an oven at 100° C. for 40 minutes. The dried beads were shaken in 3% APTES in dry toluene for 2 hours at room temperature. Then the excess reagents were rinsed off by dry toluene and the beads were treated at 100° C. until complete drying. The beads were then shaken in 2% glutaraldehyde in HPLC water for 1 hour at room temperature and the excess glutaraldehyde was removed by rinsing with HPLC grade water. 1 mg/ml picoline borane solution was prepared in 5% ethanol in HPLC water as coupling buffer, and then 80 μg/mL sdAb solution was added. The beads were shaken overnight in the coupling buffer at 4° C. followed by rinsing with PBS, and stored at 4° C. until further processing. Non-specific binding sites were blocked by incubating the beads in 10 mg/mL polyethylene-glycol (MW 8000) in PBS at 4° C. for four hours before use.

### Chemical Cleaning and Silanization of Glass Surface to Prepare Glass Beads for Anti-EpCAM Antibody Binding

Commercially available monodisperse 400 μm soda-lime glass beads were purchased from Glass Sphere (Jablonec nad Nisou, Czech Republic), microscope slides were obtained from VWR (Radnor, PA). The glass beads were pretreated with a 1:1 mixture of cc. hydrochloric acid and abs. MeOH for 30 minutes at room temperature, followed by rinsing with HPLC grade water. After the washing step, the beads were soaked in cc. sulfuric acid for 30 minutes at room temperature, rinsed again and boiled in HPLC grade water for 30 minutes [Cras, J. J., et al. 1999] [45]. The beads were oven-dried completely at 100° C. after boiling. Subsequently, the beads were shaken in 3% APTES in dry toluene for 2 hours. After rinsing the excess of reagents with dry toluene, beads were incubated again at 100° C. until complete drying. Effectiveness of chemical cleaning and uniformity of silanization were evaluated by contact angle measurements on glass slides, which were prepared the same way (FIG. 5).

### Anti-EpCAM Immobilization on Glass Beads

Anti-EpCAM antibodies were immobilized onto the glass beads and microscope slides as follows. Silanized glass beads were shaken in 2% glutaraldehyde in HPLC grade water for 1 hour. The beads were washed with HPLC grade water to rinse excess glutaraldehyde. Then 1 μg/μl non-labelled anti-EpCAM solution was added to 1 mg/ml 2-picoline-borane in 5% EtOH (V/V) coupling buffer. The beads were shaken overnight at 4° C. in the coupling buffer. Control beads were prepared in the same way, only human IgG1 antibodies were used instead of anti-EpCAM. Finally, the beads were washed with PBS (pH 7.4). The excess non-specific binding sites were blocked by 10 mg/ml PEG_{mw8000} in PBS. The immobilization process was investigated by FITC-conjugated anti-EpCAM immobilization and fluorescent microscopy (FIG. 5).

Protein Immobilization onto Poly(Methyl-Methacrylate) (PMMA) Surfaces

In the present invention a simple wet chemistry method is applied to surface modification of PMMA surfaces, removing any surface contamination with abs. ethanol/isopropanol and oxidation of methyl-ester groups with 20% sulfuric acid.

### Amination of PMMA Surfaces

In the present method the accessible methyl-esters of the native PMMA react with hexamethylenediamine, yielding primary amines on the surface, under basic pH conditions.

3-Aminopropyltriethoxysilane (APTES) is used for PMMA surface amination after sulfuric acid or other oxidizing treatments. APTES is dissolved in dry ethanol [Járvás, G., et al. 2018] [3], water [Vakili, M., et al. 2019] or acetonitrile [Miranda, A. et al. 2020] [12]. Amino-groups of the silanized surface and proteins are cross-linked by glutaraldehyde [Járvás, G., et al. 2018] [3] [Fixe, F., et al. 2004] [5, 7].

The activated surface is incubated with the protein of choice at 4° C., in 1 mg/mL 2-picoline borane in 5% EtOH coupling buffer [3, 13].

Non-specific binding can be decreased by blocking excess aldehyde groups with small molecular weight polyethyleneglycol (PEG) adsorption at 4° C.

### Virus Propagation

SARS-COV-2 (hCoV-19/Hungary/SRC_isolate_2/2020, Accession ID: EPI_ISL_483637) virus isolate was used for the experiments. Propagation of the viruses was carried out in VeroE6 cells (African green monkey kidney epithelial, ATCC CRL-1586) cultured in DMEM cell culture media containing 10% heat inactivated fetal bovine serum. Cells were incubated at 37° C. in humidified air supplied with 5% CO₂.

### Virus Capture

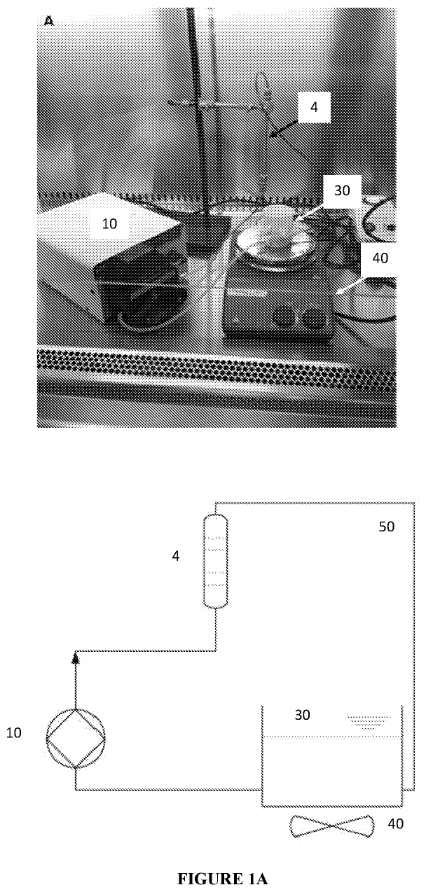

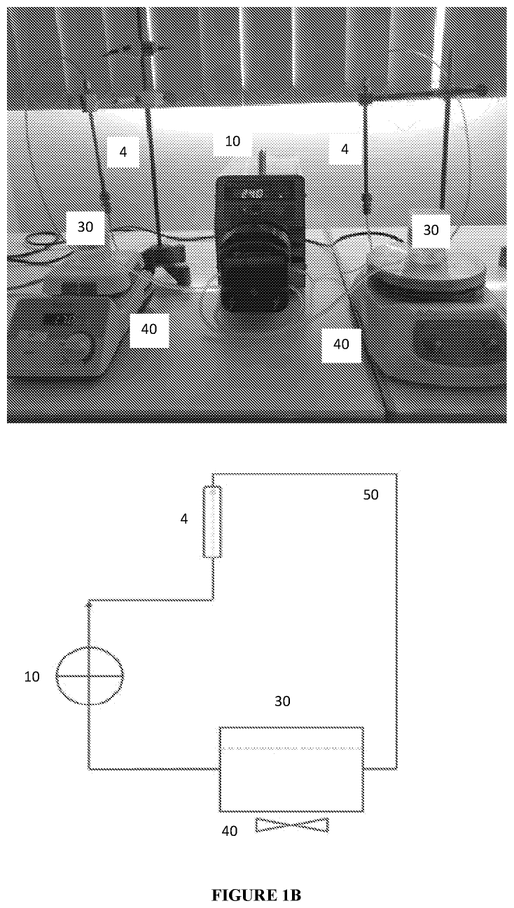

All virus culturing and capture experiments were carried out in BSL4 environment. Standard stainless steel HPLC columns (length: 16.5 cm, I.D. 4.6 mm) were modified to obtain laboratory scaled test cartridges. 400 μm hole size meshes were placed in the Swagelok fittings to retain the beads during circulation. 4 g of anti-SARS-COV-2 sdAb immobilized beads were loaded into the cartridges for the virus capture experiment. The viruses, at average ˜350 virus genome copies/μl concentration, were spiked into 30 ml DMEM medium and the virus suspension was circulated through the cartridges using a Masterflex peristaltic pump (Cole-Parmer, Vernon Hills, IL). An open lid vessel was inserted in the circulation system to equilibrate the fluid level and ensure bubble free flow. Furthermore, the vessel served as injection and sampling point as well. The schematics and photo of the experimental setup are shown in FIG. 1. The virus suspension was continuously homogenized during the experiments using a magnetic stirrer. The flow rate was set to 5 ml/min (corresponding to 155 cm/min linear velocity). To monitor any non-specific capturing of virus particles, a control experiment was also performed. The setup of the control experiment was identical as described above, but only the linker (i.e., glutaraldehyde) was immobilized onto the bead surface, and the non-specific binding sites (“capturing sites”) were masked by 10 mg/ml polyethylene-glycol (MW 8000) in PBS. The virus suspension was circulated continuously for two hours and after the circulation was stopped, the column was rinsed with PBS and the beads were transferred to falcon tubes. Virus particles were detached from the surface of the beads by washing with 1 ml 1× trypsin-EDTA solution in PBS for five minutes. Then, 2 ml PBS was added before further incubation for 5 minutes at 37° C. The bead-virus particle suspension was vortexed thoroughly, then the beads were allowed to sink and the supernatants were sampled.

On FIG. 1 a laboratory scale system according to the invention is shown with the test cartridge (FIG. 1.A) and its schematic representation (FIG. 1.B). In this embodiment the circulation is supported by a peristaltic pump (1). Cartridge (2) and vessel (3) are parts of the system and are connected with 5 tubing. The virus suspension in the vessel was homogenized by a magnetic stirrer (4).

### Quantitative Virus Genome Copy Determination

Duplicated samples of 100 μl were taken from the vessel as well as from the supernatants to determine the initial virus concentration and the number of captured virion particles, respectively. The number of captured virus particles was quantitatively determined by droplet digital PCR (QX200 Droplet Digital PCR, Bio-Rad). The Bio-Rad QuantaSoft software was used to evaluate the results. Nucleic acid isolation from the samples was performed using Monarch Total RNA Miniprep Kit (New England Biolabs, Ipswich, MA) according to the vendor's instruction to quantify viral RNA.

### Cell Culturing to Produce EpCAM Producing Cells

Non-labelled HCT116 cells were supplied by National Institute of Oncology (Budapest, Hungary) and the Hungarian Academy of Sciences (Budapest, Hungary). Cells were cultured in RPMI media containing 11% fetal bovine serum and 1% penicillin-streptomycin at 37° C. in 5% CO₂ atmosphere. Colorectal cells were proved to be EpCAM positive [Maetzel, D., et al. 2009; Lugli A. et al. 2010] [46, 47], but EpCAM expression of the cells was investigated by FITC-labelled anti-EpCAM conjugation as well (FIG. 2). Cultured cells were detached from flask walls with 1× trypsin-EDTA in PBS solution 1-2 hours prior to experiments, so the surface of the cells could regenerate before capture experiments. The detached cells were suspended in RPMI media and stored in Eppendorf tubes in the incubator at 37° C. in 5% CO₂ atmosphere.

### Flow Cytometry

100 μl cell suspension was sampled three times from the supernatant. Each sample was injected into the flow cytometer in triplicates. To increase the accuracy of quantitative cell count measurements in PBS-cell suspensions, 100 μl of FlowCount Fluorospheres internal standard was added to each sample. In this way, absolute cell counts were measured by a Gallios Flow Cytometer (Beckman Coulter, Brea, CA) enabling forward scatter (FSC), side scatter (SSC) and FL1 (green fluorescent) detectors.

### Anticoagulation

There are publications reporting about heparin anticoagulants altering antigen-antibody binding [Datta-Mannan, A., et al. 2015; Kraft, T. E. 2020] [48, 49]. The capturing efficiency of the activated beads were tested next to low molecular weight heparin (LMWH) and Na-citrate, which anticoagulants are proved to be safe in case of cancer patients [Wojtukiewicz, M. Z., et al. 2020; Moura, E. I. M., et al. 2021] [50, 51]. The dosage of anticoagulants was calculated according to the recommendations used in clinical practice [Kato, C., et al. 2021] and biological half-life of heparin also taken in consideration during experiments. During some of the experiments heparin priming was used. The experimental system was primed with 10000 IU/L Heparibene Na solution in PBS, including the test cartridge, the tank and the tubing. Priming with heparin solution before treatments is necessary in order to avoid clogging locally by the anticoagulant adhering to the surface of the extracorporeal circulation [Kato, C., et al. 2021] [52]. The selected anticoagulants were pipetted directly into the carrier medium in the calculated dosages. The effect of the anticoagulants on specific capture was evaluated by flow cytometry (FIG. 4).

### Flow Rate

Shear stress from flow rate can effect capture and viability of CTCs and CTC clusters [20, 53-56]. Specific capture of modified beads was investigated at different flow rates, according to clinical recommendations for extracorporeal devices [Dominik, A. and J. Stange, 2021; Ronco, C., et al. 2021; Naorungroj, T., et al. 2021] [57-59] (FIG. 4). Different flow rates were set and maintained by peristaltic pump (FIG. 1).

### Cell Capture

Laboratory scale test cartridges were loaded with anti-EpCAM immobilized glass beads (3.6 g of id=800 μm or 4 g of id=400 μm) for cell capture experiments. HCT116 cells, at average 18/μl concentration, were spiked into 30 ml 2% (m/V) PEG_{MW8000} in PBS. The model solution were run through the laboratory scale test system using a LeadFluid peristaltic pump (Baoding Lead Fluid Technology Co Ltd., Baoding, Hebei, China). Experimental setup and its schematics are shown in FIG. 1. As non-specific binding of adhesive cells cannot be excluded, control experiments were performed to establish a reference for accurate estimation of specific cell capture. The setup of the control experiments was identical as described above, except human IgG1 antibodies were immobilized onto the surface of the beads during the activation step instead of anti-EpCAM. Model solutions were run through the cartridge in a way that the total volume was circulated continuously for 2 hours. The cartridges were washed with PBS and the beads were transferred to 15 ml falcon tubes. Cells were detached from the beads with 1 ml 1× trypsin-EDTA solution at 37° C. for 5 minutes, then 2 ml PBS was added to each tubes and beads were incubated for another 5 minutes at the same temperature. The final volume of the cell suspensions washed from the beads were 3 ml in each tube. After the second incubation step the bead-cells suspensions were vortexed thoroughly at low rate. Then the beads were allowed to sink to the bottom of the falcon tube and supernatants were sampled. The cell concentrations were measured by flow cytometer as described above. Captured cells were also examined by fluorescent microscopy (FIG. 5).

### Scanning Electron Microscopy

Captured virus particles were visualized using scanning electron microscopy (FEI/ThermoFisher Apreo S LoVac). Observation by STEM was carried out in transmission mode under high-vacuum with 30 kV accelerating voltage. Samples were observed without fixation by following standard air-drying procedure [Adams, J. R. and T. A. Wilcox 1982] [45]. UV-C inactivated virus stock was purchased from RoLink Biotechnology (Pecs, Hungary). First, the virus suspension was dried onto a carbon stabilized formvar coated side-grid (SFR, Toronto, Canada) at room temperature for 20 minutes to determine its morphology. Then, glass beads with captured virus particles on their surface were took out from the stainless steel cartridge after a regular capture cycle. To remove any remaining residues from the culture media, beads were rinsed with HPLC grade water. Low-vacuum mode was used with 10 kV accelerating voltage to detect the virus on the surface of the glass beads.

### Example 2—Results of Virus Capturing

In this Example a high throughput extracorporeal device has been designed to specifically remove virions from human cardiovascular circulation with the goal to avoid serious clinical outcome. A laboratory scale hemoperfusion system has been fabricated to study the feasibility of the proposed approach, which combines the specificity of affinity chromatography with high throughput of hemoperfusion technique. The scheme of the proposed virus capture technology is shown in FIG. 2 showing a schematic representation of the virus capture mechanism. In the exemplary embodiment shown the immobilized nanobodies specifically bound the spike protein of SARS-COV-2 particles, thus, selectively capture them from the blood stream.

Capture efficiency was investigated using an actual strain of SARS-COV-2 in a BSL-4 classified laboratory. In the presented proof-of-concept experiments, the virus particles were captured from a culture medium containing L-glutamine, sodium pyruvate, sodium bicarbonate, and phenol red indicator. Albeit, its complexity lags behind that of blood complexity, it can adequately model the virus capture efficiency from fluidic circulation. Virus concentrations were determined by ddPCR, both in the active and control experiments (Table 1).

TABLE 1
Results of the ddPCR analysis. The reported virus copy
values represent the genomic concentrations. SARS-CoV-2
virus particles were spiked into PBS buffer, followed
by virus capture utilizing the technology proposed in
this paper. All experiments were done in duplicates.

| | | Initial | Leftover | Eluted |
|---|---|---|---|---|
| | | [copy/μl] | | |
| Active | Sample 1 | 364 | 23 | 129 |
| | Sample 2 | 331 | 29 | 117 |
| | Average | 348 | 26 | 123 |
| Control | Sample 1 | 303 | 102 | 73 |
| | Sample 2 | 397 | 108 | 93 |
| | Average | 350 | 105 | 83 |

As apparent from Table 1, virus particles must have been non-specifically bound to the plastic components of the equipment (buffer vessel, tubing and connectors) and on the surface of the glass beads, represented by the difference between the input-(leftover+eluted).

However, it should be emphasized that the surface-to-volume ratio of this laboratory scale experimental setup was significantly higher than that of in an actual hemoperfusion device, thus non-specific binding was overrepresented. The measure of non-specific binding was determined by balancing the captured virus copies in the active and control experiments. Note, only those virus particles were considered during the evaluation, which remained in the circulation and did not bind non-specifically, i.e., not removed from the circulation. During the two-hour experiment, the active cartridge captured 32% of the virus particles from the circulation, while the control cartridge was able to remove only 7%, apparently representing non-specific binding.

After the capture efficiency studies, scanning electron microscope (SEM) imaging was utilized to visualize the captured viruses (images of virions) on the surface of the beads. To this end, ˜4·10⁵ PFU/ml concentration virus suspension was dried onto a microscope slide-grid to observe certain morphologies of the virions under the conditions of the actual experimental setup of this example, see FIGS. 3.A and 3.B. FIGS. 3.C and 3.D show the captured virus particles (virions) on the surface of the beads. Images were taken using a FEI Quanta 3D FEG instrument.

Please note that in this Example the same capturing method was used as for the efficiency measurements above, but the captured virus particles were rinsed with HPLC grade water to remove PBS residues in order to improve image sharpness. After SEM imaging the beads were trypsin-EDTA washed for verifying the microscopy observations. The existence of SARS-COV-2 virions in the eluent was successfully verified by conventional qPCR method.

### Example 3—Clinical Treatment for Virus Cleansing

According to current clinical protocols, similar extracorporeal treatments may last up to 24 hours [Peng, J. Y., et al. 2021] [46], that, based on our results, would readily capture practically all circulating virus particles. Both during the active and control experiments, the captured virus particles were eluted from the surface of the glass beads by buffer rinse with trypsin wash. Comparing the active and control experiments, the difference in the number of specifically captured viruses was considered to be the actual performance, i.e., found to be 120,000 virus copies.

### Example 4—Extracorporeal Method for Capturing CTCs-Results

In this study, we report on the modification of commercially available glass beads for high volume specific removal of cells from a laboratory test scale system. Specific capture from buffer model solution was determined by flow cytometry. The scheme of the proposed technology is shown in FIG. 2.b

EpCAM (CD326) expression of HCT116 cell line was confirmed by FITC-labelled anti-EpCAM conjugation. Fluorescence of the cell-antibody conjugates was ascertained by flow cytometry. The histograms of cytometry measurements are shown in FIG. 4. Following successful conjugation, uniform green fluorescence of the HCT116 cells clearly demonstrates the EpCAM expression of the colon cell line.

Goniometry was used to examine the quality of the prepared glass surfaces. Glass slides were chemically cleaned and silanized as described above. Efficiency of each step were examined after drying under laminar hood. Evaluation of contact angles was carried out by FTA32 software (FIG. 5). After chemical cleaning, the number of hydroxyl-groups on the surface of the glass increases raising hydrophilicity, resulting in the decrease of the contact angle significantly compared to the initial state. APTES molecules bind to these hydroxyl groups, increasing the hydrophobicity of the surface, which causes an increase in the contact angle.

To determine the capture efficiency of the activated beads, the cells obtained after the trypsin-EDTA washing were taken into consideration. The cultured HCT116 cells are tend to attach to the walls of the tank and the tubing, therefore, the concentration of cells remaining in the carrier medium did not provide accurate information that could be used to calculate efficacy, thus the cells actually captured by the beads were taken into consideration. Specific capture of the beads was used to characterize efficiency. Specific capture was calculated according to the following. After trypsinization, the detached cells from the activated and control beads were quantitatively measured by flow cytometer. The difference in the number of cells washed from the active and control beads refers to the cells that were specifically captured from the carrier medium by the immobilized anti-EpCAM molecules on the surface of the activated beads [captured cells_{(activated beads)}-nonspecific capture_{(control beads)}].

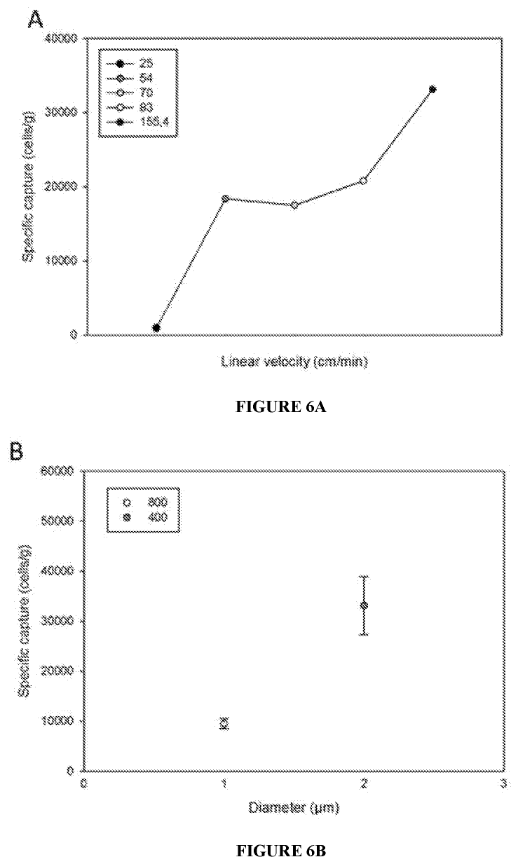

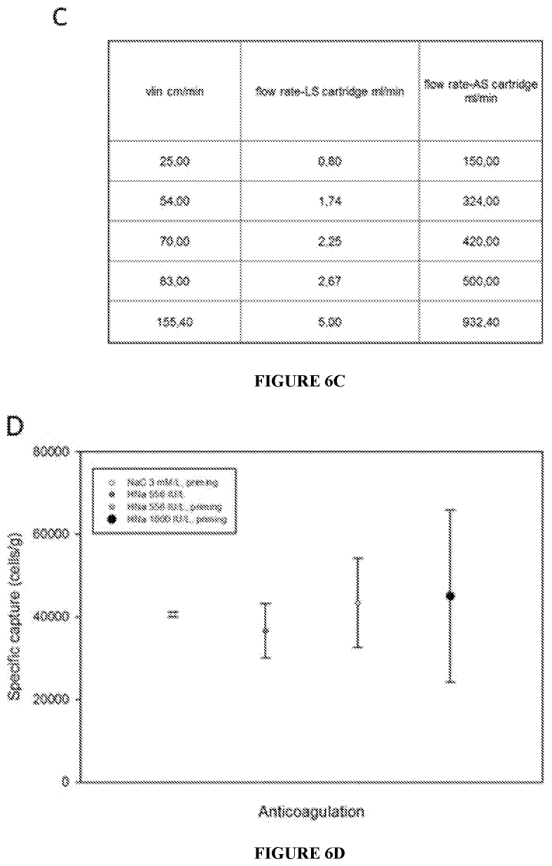

The results of the capture experiments are shown in FIG. 6.

Decrease in bead size, i.e., increase in specific surface area was investigated by capture experiments. The filling weight of the identical laboratory scale cartridges was 3.5 g in case of the 800 μm diameter beads and 4 g in case of the 400 μm diameter beads. Significant increase in specific capture was observed with increased surface area, from an average of 34,000 cells to over 130,000 cells next to 5 ml/min flow rate. The laboratory scale cartridge contains approximately 2.6 cm³ glass beads, thus based on the results of the present experiments, specific capture of a device with a volume of 330 cm³ even could reach 16.5 million cells. An average cancer patient's 7.5 ml blood contains ˜5 cells in case of a poor prognosis, which is significantly lower than the capturing ability of the anti-EpCAM immobilized glass beads. Based on these results, the experiments were continued with 400 μm diameter beads (FIG. 6).

Specific capture of activated beads was also examined at different linear velocities. The applied flow parameter range was designed according to clinical recommendation for extracorporeal treatments. The actual flow rates were calculated considering the dimensions of the CytoSorb® hemoperfusion cartridge [Friesecke, S., et al. 2019] [60], since the size polymer beads used in this device are similar to the glass beads, that were used during cell capture experiments. The calculated linear velocities and flow rates are shown in FIG. 6. Specific capture increases with increasing linear velocity. This is possible because the non-specific binding of the cells decreases as a result of the accelerating flow, but the cells are still able to specifically bind to the immobilized anti-EpCAM molecules, thus for a 330 cm³ device, a flow rate of more than 500 ml/min is suggested.

Interaction of immobilized antibodies and anticoagulants was also investigated (FIG. 6). It can be concluded that anticoagulant do not affect specific capture negatively, however, with increasing heparin concentration, the results of parallel capture experiments show large variance, but negligible in terms of the effectiveness of the activated beads.

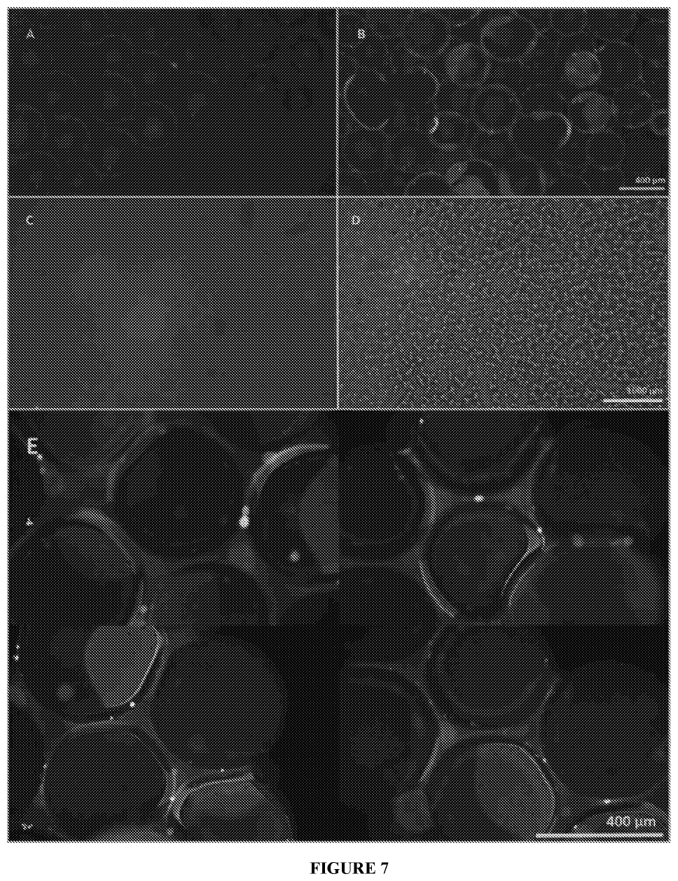

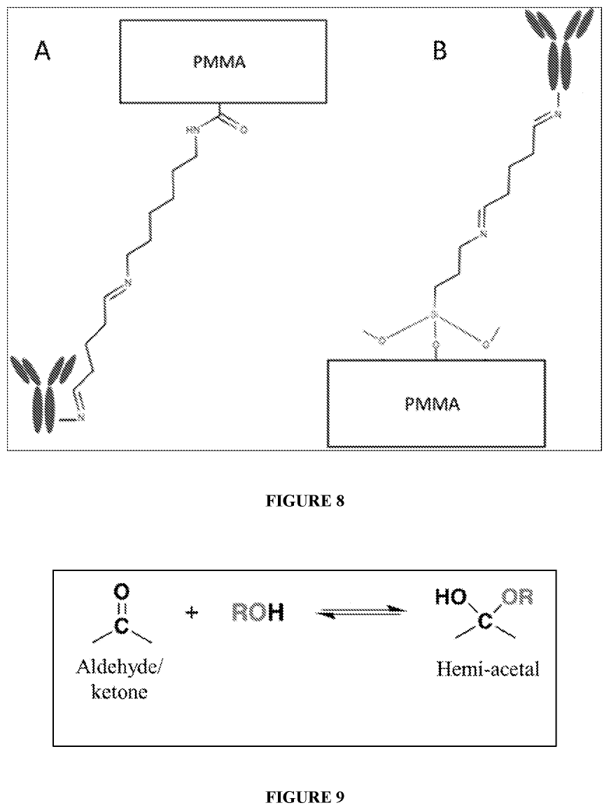

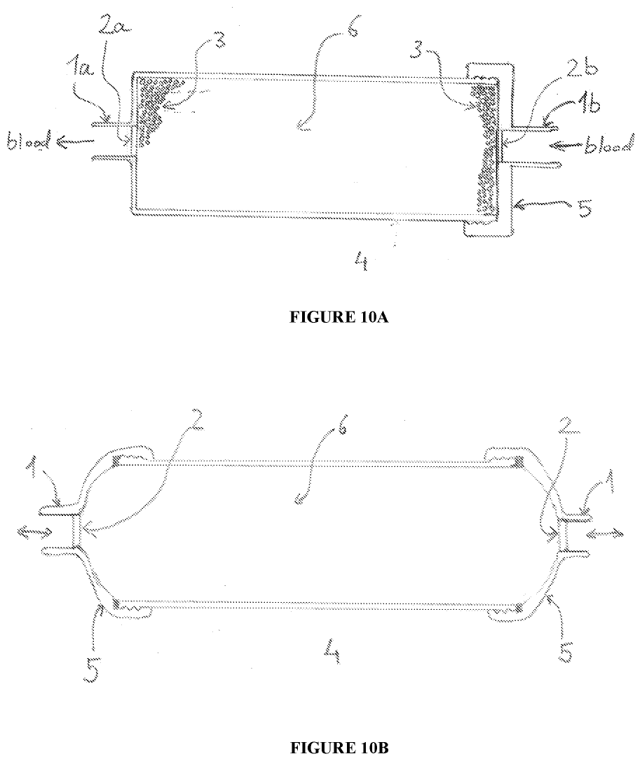

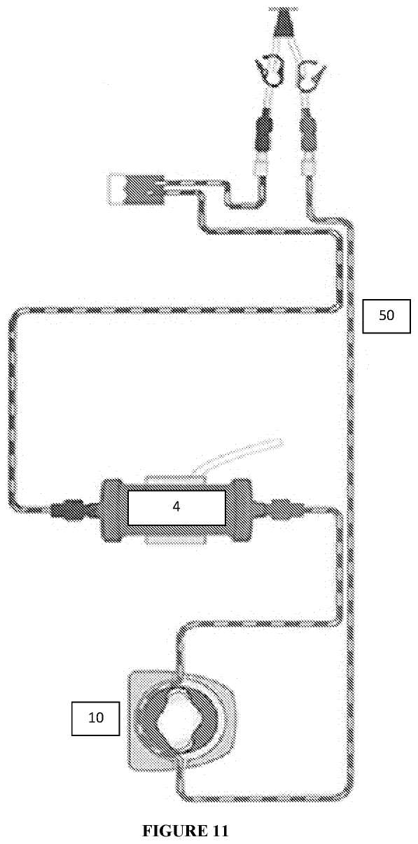

Successful immobilization of antibodies and bead functionality were also examined by fluorescent microscopy. To clearly show the difference between active and control surfaces, anti-EpCAM molecules were immobilized not only onto glass beads, but also on glass slides, and cells were labelled with calcein-AM (FIG. 7). Captured cells are visible in panels B and C. In panel A there are only a few cells bound non-specifically to the surface of the control glass slide.

REFERENCES

| | |
|---|---|
| Wu, F., et al., A new coronavirus associated with human respiratory disease in China. | 1. |
| 2020. 579(7798): p. 265-269. | |
| Zhou, P., et al., A pneumonia outbreak associated with a new coronavirus of probable | 2. |
| bat origin. Nature, 2020. 579(7798): p. 270-273. | |
| Haldane, V., et al., Health systems resilience in managing the COVID-19 pandemic: | 3. |
| lessons from 28 countries. Nature Medicine, 2021. 27(6): p. 964-980. | |
| Kissler, S. M. and C. Tedijanto, Projecting the transmission dynamics of SARS-CoV-2 | 4. |
| through the postpandemic period. 2020. 368(6493): p. 860-868. | |
| Carvalho, T., F. Krammer, and A. Iwasaki, The first 12 months of COVID-19: a | 5. |
| timeline of immunological insights. Nature Reviews Immunology, 2021. 21(4): p. 245- | |
| 256. | |
| Weissleder, R., et al., COVID-19 diagnostics in context. Science Translational | 6. |
| Medicine, 2020. 12(546): p. eabc1931. | |
| Vandenberg, O., et al., Considerations for diagnostic COVID-19 tests. Nature Reviews | 7. |
| Microbiology, 2021. 19(3): p. 171-183. | |
| Rahimi, H., et al., CRISPR Systems for COVID-19 Diagnosis. ACS Sensors, 2021. | 8. |
| 6(4): p. 1430-1445. | |
| Guo, K., et al., Rapid single-molecule detection of COVID-19 and MERS antigens via | 9. |
| nanobody-functionalized organic electrochemical transistors. Nature Biomedical | |
| Engineering, 2021. 5(7): p. 666-677. | |
| Tang, Z., et al., A materials-science perspective on tackling COVID-19. Nature | 10. |
| Reviews Materials, 2020. 5(11): p. 847-860. | |
| Sheridan, C., COVID-19 spurs wave of innovative diagnostics. Nature Biotechnology, | 11. |
| 2020. 38(7): p. 769-772. | |
| Mullard, A., COVID-19 vaccine development pipeline gears up. Lancet, 2020. | 12. |
| 395(10239): p. 1751-1752. | |
| Swol, J. and R. Lorusso, Additive treatment considerations in COVID-19-The | 13. |
| clinician's perspective on extracorporeal adjunctive purification techniques. Artificial | |
| Organs, 2020. 44(9): p. 918-925. | |
| Xi, Y., Convalescent plasma therapy for COVID-19: a tried-and-true old strategy? | 14. |
| Signal Transduction and Targeted Therapy, 2020. 5(1): p. 203. | |
| Subbarao, K., F. Mordant, and R. Rudraraju, Convalescent plasma treatment for | 15. |
| COVID-19: Tempering expectations with the influenza experience. European Journal | |
| of Immunology, 2020. 50(10): p. 1447-1453. | |
| Jeyanathan, M., et al., Immunological considerations for COVID-19 vaccine strategies. | 16. |
| Nature Reviews Immunology, 2020. 20(10): p. 615-632. | |
| Graham, B. S., Rapid COVID-19 vaccine development. Science, 2020. 368(6494): p. | 17. |
| 945-946. | |
| Tao, K., et al., The biological and clinical significance of emerging SARS-CoV-2 | 18. |
| variants. Nature Reviews Genetics, 2021. | |
| Rasmussen, A. L. and S. V. Popescu, SARS-CoV-2 transmission without symptoms. | 19. |
| Science, 2021. 371(6535): p. 1206-1207. | |
| Chang, M. C., et al., Risk factors for disease progression in COVID-19 patients. BMC | 20. |
| Infectious Diseases, 2020. 20(1): p. 445. | |
| Ayres, J. S., A metabolic handbook for the COVID-19 pandemic. Nature Metabolism, | 21. |
| 2020. 2(7): p. 572-585. | |
| Richardson, S., et al., Presenting Characteristics, Comorbidities, and Outcomes Among | 22. |
| 5700 Patients Hospitalized With COVID-19 in the New York City Area. Jama, 2020. | |
| 323(20): p. 2052-2059. | |
| Varga, Z., et al., Endothelial cell infection and endotheliitis in COVID-19. The Lancet, | 23. |
| 2020. 395(10234): p. 1417-1418. | |
| Fajnzylber, J., et al., SARS-CoV-2 viral load is associated with increased disease | 24. |
| severity and mortality. Nature Communications, 2020. 11(1): p. 5493. | |
| Pujadas, E., et al., SARS-CoV-2 viral load predicts COVID-19 mortality. Lancet Respir | 25. |
| Med, 2020. 8(9): p. e70. | |
| Tsukagoshi, H., et al., Relationships between Viral Load and the Clinical Course of | 26. |
| COVID-19. Viruses, 2021. 13(2): p. 304. | |
| Cao, W. and T. Li, COVID-19: towards understanding of pathogenesis. Cell Research, | 27. |
| 2020. 30(5): p. 367-369. | |
| Müller, J. A., et al., SARS-CoV-2 infects and replicates in cells of the human endocrine | 28. |
| and exocrine pancreas. Nature Metabolism, 2021. 3(2): p. 149-165. | |
| Perotti, C. and C. Del Fante, Plasma from donors recovered from the new Coronavirus | 29. |
| 2019 as therapy for critical patients with COVID-19 (COVID-19 plasma study): a | |
| multicentre study protocol. 2020. 15(5): p. 819-824. | |
| Xu, Z., et al., Pathological findings of COVID-19 associated with acute respiratory | 30. |
| distress syndrome. Lancet Respir Med, 2020. 8(4): p. 420-422. | |
| Hagman, K., et al., SARS-CoV-2 RNA in serum as predictor of severe outcome in | 31. |
| COVID-19: a retrospective cohort study. Clin Infect Dis, 2020. | |
| Eberhardt, K. A., et al., RNAemia Corresponds to Disease Severity and Antibody | 32. |
| Response in Hospitalized COVID-19 Patients. 2020. 12(9). | |
| Xu, D., et al., Relationship Between Serum Severe Acute Respiratory Syndrome | 33. |
| Coronavirus 2 Nucleic Acid and Organ Damage in Coronavirus 2019 Patients: A | |
| Cohort Study. 2021. 73(1): p. 68-75. | |
| Barbaro, R. P., et al., Extracorporeal membrane oxygenation for COVID-19: evolving | 34. |
| outcomes from the international Extracorporeal Life Support Organization Registry. | |
| The Lancet, 2021. 398(10307): p. 1230-1238. | |
| Ronco, C., et al., Extracorporeal Blood Purification and Organ Support in the Critically | 35. |
| Ill Patient during COVID-19 Pandemic: Expert Review and Recommendation. Blood | |
| Purification, 2021. 50(1): p. 17-27. | |
| Ramírez-Guerrero, G., et al., Early Cytokine Removal in Critical COVID-19 Patients | 36. |
| with Extracorporeal Therapies (HA-380 plus High Volume Hemofiltration) May | |
| Prevent Progression of Acute Respiratory Distress Syndrome: Case Report. Blood | |
| Purification, 2021. 50(4-5): p. 575-577. | |
| Shadvar, K., et al., Hemoperfusion as a Potential Treatment for Critically Ill COVID- | 37. |
| 19 Patients with Cytokine Storm. Blood Purification, 2021. 50(3-4): p. 405-407. | |
| Seffer, M. T., et al., Heparin 2.0: A New Approach to the Infection Crisis. Blood | 38. |
| Purification, 2021. 50(1): p. 28-34. | |
| Pape, A., et al., Treatment of a Critically Ill COVID-19 Patient with the Seraph 100 | 39. |
| Microbind Affinity Filter. TH Open, 2021. 05(02): p. e134-e138. | |
| Amundson, D. E., et al., Removal of COVID-19 Spike Protein, Whole Virus, | 40. |
| Exosomes, and Exosomal MicroRNAs by the Hemopurifier ® Lectin-Affinity | |
| Cartridge in Critically Ill Patients With COVID-19 Infection. Frontiers in Medicine, | |
| 2021. 8(1763). | |
| Wrapp, D., et al., Structural Basis for Potent Neutralization of Betacoronaviruses by | 41. |
| Single-Domain Camelid Antibodies. Cell, 2020. 181(5): p. 1004-1015.e15. | |
| Reider, B., et al., Integrated workflow for urinary prostate specific antigen N- | 42. |
| glycosylation analysis using sdAb partitioning and downstream capillary | |
| electrophoresis separation. Analytica Chimica Acta, 2021. 1184: p. 338892. | |
| Meszaros, B., et al., N-glycomic Analysis of Z(IgA1) Partitioned Serum and Salivary | 43. |
| Immunoglobulin A by Capillary Electrophoresis. Current Molecular Medicine, 2020. | |
| 20(10): p. 781-788. | |
| Cras, J. J., et al., Comparison of chemical cleaning methods of glass in preparation for | 44. |
| silanization. Biosensors and Bioelectronics, 1999. 14(8): p. 683-688. | |
| Adams, J. R. and T. A. Wilcox, Scanning electron microscopical comparisons of insect | 45. |
| virus occlusion bodies prepared by several techniques. Journal of Invertebrate | |
| Pathology, 1982. 40: p. 12-20. | |
| Peng, J. Y., et al., Hemoperfusion with CytoSorb ® in Critically Ill COVID-19 Patients. | 46. |
| Blood Purification, 2021. | |
| Vivanti, A. J., et al., Transplacental transmission of SARS-CoV-2 infection. Nature | 47. |
| Communications, 2020. 11(1): p. 3572. | |
| Colagrossi, L., et al., SARS-CoV-2 RNA in plasma samples of COVID-19 affected | 48. |
| individuals: a cross-sectional proof-of-concept study. BMC Infectious Diseases, 2021. | |
| 21(1): p. 184. | |
| van Doremalen, N., et al., Intranasal ChAdOx1 nCoV-19/AZD1222 vaccination | 49. |
| reduces viral shedding after SARS-CoV-2 D614G challenge in preclinical models. Sci | |
| Transl Med, 2021. 13(607). | |
| Israelow, B., et al., Adaptive immune determinants of viral clearance and protection in | 50. |
| mouse models of SARS-CoV-2. Sci Immunol, 2021. 6(64): p. eabl4509. | |
| Ashworth, T. R., A case of cancer in which cells similar to those in the tumours were | 1. |
| seen in the blood after death. Aust Med J., 1869. 14: p. 146. | |
| Brisotto, G., et al., Dysmetabolic Circulating Tumor Cells Are Prognostic in Metastatic | 2. |
| Breast Cancer. Cancers (Basel), 2020. 12(4). | |
| Wu, M., et al., Circulating Tumor Cell Phenotyping via High-Throughput Acoustic | 3. |
| Separation. Small, 2018. 14(32): p. e1801131. | |
| Schochter, F., et al., Are Circulating Tumor Cells (CTCs) Ready for Clinical Use in | 4. |
| Breast Cancer? An Overview of Completed and Ongoing Trials Using CTCs for | |
| Clinical Treatment Decisions. Cells, 2019. 8(11). | |
| Tayoun, T., et al., CTC-Derived Models: A Window into the Seeding Capacity of | 5. |
| Circulating Tumor Cells (CTCs). Cells, 2019. 8(10). | |
| Keup, C., et al., Multimodal Targeted Deep Sequencing of Circulating Tumor Cells and | 6. |
| Matched Cell-Free DNA Provides a More Comprehensive Tool to Identify Therapeutic | |
| Targets in Metastatic Breast Cancer Patients. Cancers (Basel), 2020. 12(5). | |
| Cimadamore, A., et al., Update on Circulating Tumor Cells in Genitourinary Tumors | 7. |
| with Focus on Prostate Cancer. 2020. 9(6). | |
| Mamdouhi, T., et al., Fugitives on the run: circulating tumor cells (CTCs) in metastatic | 8. |
| diseases. Cancer and Metastasis Reviews, 2019. 38(1): p. 297-305. | |
| Chicón-Bosch, M. and O. M. Tirado, Exosomes in Bone Sarcomas: Key Players in | 9. |
| Metastasis. Cells, 2020. 9(1). | |
| Yang, B., et al., High-metastatic cancer cells derived exosomal miR92a-3p promotes | 10. |
| epithelial-mesenchymal transition and metastasis of low-metastatic cancer cells by | |
| regulating PTEN/Akt pathway in hepatocellular carcinoma. Oncogene, 2020. 39(42): | |
| p. 6529-6543. | |
| Ignatiadis, M. and G. W. Sledge, Liquid biopsy enters the clinic - implementation issues | 11. |
| and future challenges. 2021. 18(5): p. 297-312. | |
| Alix-Panabières, C. and K. Pantel, Liquid Biopsy: From Discovery to Clinical | 12. |
| Application. Cancer Discovery, 2021. 11(4): p. 858-873. | |
| Cescon, D. W., et al., Circulating tumor DNA and liquid biopsy in oncology. Nature | 13. |
| Cancer, 2020. 1(3): p. 276-290. | |
| Rushton, A. J., et al., A Review of Circulating Tumour Cell Enrichment Technologies. | 14. |
| Cancers (Basel), 2021. 13(5). | |
| Alix-Panabières, C., “Circulating Tumor Cells: Finding Rare Events for a Huge | 15. |
| Knowledge of Cancer Dissemination”. Cells, 2020. 9(3). | |
| Ribeiro-Samy, S., et al., Fast and efficient microfluidic cell filter for isolation of | 16. |
| circulating tumor cells from unprocessed whole blood of colorectal cancer patients. | |
| Scientific Reports, 2019. 9(1): p. 8032. | |
| Adams, D. L., et al., Cytometric characterization of Circulating Tumor Cells Captured | 17. |
| by microfiltration and their correlation to the cellsearch ® CTC test. Cytometry Part A, | |
| 2015. 87(2): p. 137-144. | |
| Gorin, M. A., et al., Circulating tumour cells as biomarkers of prostate, bladder, and | 18. |
| kidney cancer. Nat Rev Urol, 2017. 14(2): p. 90-97. | |
| Lee, Y., G. Guan, and A. A. Bhagat, ClearCell ® FX, a label-free microfluidics | 19. |
| technology for enrichment of viable circulating tumor cells. Cytometry A, 2018. | |
| 93(12): p. 1251-1254. | |
| Sarioglu, A. F., et al., A microfluidic device for label-free, physical capture of | 20. |
| circulating tumor cell clusters. Nature Methods, 2015. 12(7): p. 685-691. | |
| Edd, J. F., et al., Microfluidic concentration and separation of circulating tumor cell | 21. |
| clusters from large blood volumes. Lab on a Chip, 2020. 20(3): p. 558-567. | |
| Gaitas, A. and G. Kim, Chemically Modified Plastic Tube for High Volume Removal | 22. |
| and Collection of Circulating Tumor Cells. PLoS One, 2015. 10(7): p. e0133194. | |
| Kang, J. H., et al., An extracorporeal blood-cleansing device for sepsis therapy. Nature | 23. |
| Medicine, 2014. 20(10): p. 1211-1216. | |
| Kim, G. and A. Gaitas, Extracorporeal Photo-Immunotherapy for Circulating Tumor | 24 |
| Cells. PLOS ONE, 2015. 10(5): p. e0127219. | |
| Garban, F., et al., Extracorporeal photopheresis as a curative treatment strategy in non | 25. |
| epidermotropic T-cell lymphoma and large granular lymphocyte leukemia. Ann Oncol, | |
| 2012. 23(9): p. 2386-2390. | |
| Vieyra-Garcia, P. A. and P. Wolf, Extracorporeal Photopheresis: A Case of | 26. |
| Immunotherapy Ahead of Its Time. Transfusion Medicine and Hemotherapy, 2020. | |
| 47(3): p. 226-235. | |
| Darvekar, S., et al., Selective Killing of Activated T Cells by 5-Aminolevulinic Acid | 27. |
| Mediated Photodynamic Effect: Potential Improvement of Extracorporeal | |
| Photopheresis. Cancers (Basel), 2020. 12(2). | |
| Kim, G., H. Vinerean, and A. Gaitas, A Novel Pathogen Capturing Device for Removal | 28. |
| and Detection. Scientific Reports, 2017. 7(1): p. 5552. | |
| Jarvas, G., et al., Modification of Hemodialysis Membranes for Efficient Circulating | 29. |
| Tumor Cell Capture for Cancer Therapy. Molecules, 2021. 26(16): p. 4845. | |
| Martin, O. A., et al., Does the mobilization of circulating tumour cells during cancer | 30. |
| therapy cause metastasis? Nature Reviews Clinical Oncology, 2017. 14(1): p. 32-44. | |
| Juratli, M. A., et al., Real-time monitoring of circulating tumor cell release during tumor | 31. |
| manipulation using in vivo photoacoustic and fluorescent flow cytometry. Head Neck, | |
| 2014. 36(8): p. 1207-15. | |
| Joosse, S. A., et al., Tumor-Associated Release of Prostatic Cells into the Blood after | 32. |
| Transrectal Ultrasound-Guided Biopsy in Patients with Histologically Confirmed | |
| Prostate Cancer. Clin Chem, 2020. 66(1): p. 161-168. | |
| Hiller, J. G., et al., Perioperative events influence cancer recurrence risk after surgery. | 33. |
| Nature Reviews Clinical Oncology, 2018. 15(4): p. 205-218. | |
| Bracci, L., et al., Immune-based mechanisms of cytotoxic chemotherapy: implications | 34. |
| for the design of novel and rationale-based combined treatments against cancer. Cell | |
| Death Differ, 2014. 21(1): p. 15-25. | |
| Zitvogel, L., O. Kepp, and G. Kroemer, Immune parameters affecting the efficacy of | 35. |
| chemotherapeutic regimens. Nat Rev Clin Oncol, 2011. 8(3): p. 151-60. | |
| Zitvogel, L., et al., Immunological aspects of cancer chemotherapy. Nat Rev Immunol, | 36. |
| 2008. 8(1): p. 59-73. | |
| Ghiringhelli, F. and L. Apetoh, The interplay between the immune system and | 37. |
| chemotherapy: emerging methods for optimizing therapy. Expert Rev Clin Immunol, | |
| 2014. 10(1): p. 19-30. | |
| Morrison, V. A., Immunosuppression associated with novel chemotherapy agents and | 38. |
| monoclonal antibodies. Clin Infect Dis, 2014. 59 Suppl 5: p. S360-4. | |
| Redmond, K. M., et al., Resistance mechanisms to cancer chemotherapy. Front Biosci, | 39. |
| 2008. 13: p. 5138-54. | |
| Bukowski, K., M. Kciuk, and R. Kontek, Mechanisms of Multidrug Resistance in | 40. |
| Cancer Chemotherapy. 2020. 21(9). | |
| Raguz, S. and E. Yagüe, Resistance to chemotherapy: new treatments and novel | 41. |
| insights into an old problem. British Journal of Cancer, 2008. 99: p. 387-391. | |
| Hoy, A. J., S. R. Nagarajan, and L. M. Butler, Tumour fatty acid metabolism in the | 42. |
| context of therapy resistance and obesity. Nature Reviews Cancer, 2021. | |
| Dudas, J., et al., Epithelial to Mesenchymal Transition: A Mechanism that Fuels Cancer | 43. |
| Radio/Chemoresistance. Cells, 2020. 9(2). | |
| Oshima, K., et al., Mutational and functional genetics mapping of chemotherapy | 44. |
| resistance mechanisms in relapsed acute lymphoblastic leukemia. Nature Cancer, 2020. | |
| 1(11): p. 1113-1127. | |
| Cras, J. J., et al., Comparison of chemical cleaning methods of glass in preparation for | 45. |
| silanization. Biosensors and Bioelectronics, 1999. 14(8): p. 683-688. | |
| Maetzel, D., et al., Nuclear signalling by tumour-associated antigen EpCAM. Nat Cell | 46. |
| Biol, 2009. 11(2): p. 162-71. | |
| Lugli, A., et al., Prognostic impact of the expression of putative cancer stem cell | 47. |
| markers CD133, CD166, CD44s, EpCAM, and ALDH1 in colorectal cancer. Br J | |
| Cancer, 2010. 103(3): p. 382-90. | |
| Datta-Mannan, A., et al., The interplay of non-specific binding, target-mediated | 48. |
| clearance and FcRn interactions on the pharmacokinetics of humanized antibodies. | |
| MAbs, 2015. 7(6): p. 1084-93. | |
| Kraft, T. E., W. F. Richter, and T. Emrich, Heparin chromatography as an in vitro | 49. |
| predictor for antibody clearance rate through pinocytosis. 2020. 12(1): p. 1683432. | |
| Wojtukiewicz, M. Z., et al., Direct Oral Anticoagulants in Cancer Patients. Time for a | 50. |
| Change in Paradigm. Cancers (Basel), 2020. 12(5). | |
| Moura, E. I. M., et al., Efficacy and Safety of Regional Anticoagulation with 4% | 51. |
| Trisodium Citrate Versus Heparin in Extended Hemodialysis among Critical Patients | |
| with Cancer and Acute Kidney Injury. Blood Purif, 2021. 50(1): p. 50-56. | |
| Kato, C., et al., Anticoagulation strategies in extracorporeal circulatory devices in adult | 52. |
| populations. European Journal of Haematology, 2021. 106(1): p. 19-31. | |
| Marrella, A., et al., High blood flow shear stress values are associated with circulating | 53. |
| tumor cells cluster disaggregation in a multi-channel microfluidic device. 2021. 16(1): | |
| p. e0245536. | |
| Regmi, S., A. Fu, and K. Q. Luo, High Shear Stresses under Exercise Condition Destroy | 54. |
| Circulating Tumor Cells in a Microfluidic System. Scientific Reports, 2017. 7(1): p. | |
| 39975. | |
| Li, P., et al., Probing circulating tumor cells in microfluidics. Lab Chip, 2013. 13(4): p. | 55. |
| 602-9. | |
| Murlidhar, V., et al., A radial flow microfluidic device for ultra-high-throughput | 56. |
| affinity-based isolation of circulating tumor cells. Small, 2014. 10(23): p. 4895-904. | |
| Dominik, A. and J. Stange, Similarities, Differences, and Potential Synergies in the | 57. |
| Mechanism of Action of Albumin Dialysis Using the MARS Albumin Dialysis Device | |
| and the CytoSorb Hemoperfusion Device in the Treatment of Liver Failure. Blood | |
| Purif, 2021. 50(1): p. 119-128. | |
| Ronco, C., et al., Extracorporeal Blood Purification and Organ Support in the Critically | 58 |
| Ill Patient during COVID-19 Pandemic: Expert Review and Recommendation. Blood | |
| Purification, 2021. 50(1): p. 17-27. | |
| Naorungroj, T., et al., Extracorporeal Ammonia Clearance for Hyperammonemia in | 59. |
| Critically Ill Patients: A Scoping Review. Blood Purification, 2021. 50(4-5): p. 453- | |
| 461. | |
| Friesecke, S., et al., International registry on the use of the CytoSorb ® adsorber in ICU | 60. |
| patients: Study protocol and preliminary results. Med Klin Intensivmed Notfmed, | |
| 2019. 114(8): p. 699-707. | |
| Barbosa, A. and N. Reis, A critical insight into the development pipeline of | 1. |
| microfluidic immunoassay devices for sensitive quantitation of protein biomarkers at | |
| point-of-care. The Analyst, 2017. 142. | |
| Sathish, S., N. Ishizu, and A. Q. Shen, Air Plasma-Enhanced Covalent | 2. |
| Functionalization of Poly(methyl methacrylate): High-Throughput Protein | |
| Immobilization for Miniaturized Bioassays. ACS Applied Materials & Interfaces, | |
| 2019. 11(49): p. 46350-46360. | |
| Járvás, G., et al., Tilted pillar array fabrication by the combination of proton beam | 3. |
| writing and soft lithography for microfluidic cell capture Part 2: Image sequence | |
| analysis based evaluation and biological application. ELECTROPHORESIS, 2018. | |
| 39(3): p. 534-539. | |
| Wardani, A. K., et al., Preparation of hydrophilic polypropylene membrane by acid | 4. |
| dipping technique. Materials Research Express, 2019. 6(7): p. 075308. | |
| Fixe, F., et al., Functionalization of poly(methyl methacrylate) (PMMA) as a substrate | 5. |
| for DNA microarrays. Nucleic Acids Research, 2004. 32(1): p. e9-e9. | |
| Jung, Y., J. Y. Jeong, and B. H. Chung, Recent advances in immobilization methods of | 6. |
| antibodies on solid supports. Analyst, 2008. 133(6): p. 697-701. | |
| Fixe, F., et al., Functionalization of poly(methyl methacrylate) (PMMA) as a substrate | 7. |
| for DNA microarrays. Nucleic Acids Res, 2004. 32(1): p. e9. | |
| Cheng, J.-Y., et al., Direct-write laser micromachining and universal surface | 8. |
| modification of PMMA for device development. Sensors and Actuators B: Chemical, | |
| 2004. 99(1): p. 186-196. | |
| Rezaei, F., B. Shokri, and M. Sharifian, Atmospheric-pressure DBD plasma-assisted | 9. |
| surface modification of polymethyl methacrylate: A study on cell growth/proliferation | |
| and antibacterial properties. Applied Surface Science, 2016. 360: p. 641-651. | |
| Acsente, T., et al., Surface modification of polymethylmethacrylate foils using an | 10. |
| atmospheric pressure plasma jet in presence of water vapors. Thin Solid Films, 2016. | |
| 614: p. 25-30. | |
| Vakili, M., et al., Ultrasound-Assisted Preparation of Chitosan/Nano-Activated Carbon | 11. |
| Composite Beads Aminated with (3-Aminopropyl)Triethoxysilane for Adsorption of | |
| Acetaminophen from Aqueous Solutions. Polymers, 2019. 11(10): p. 1701. | |
| Miranda, A., L. Martínez, and P. A. A. De Beule, Facile synthesis of an | 12. |
| aminopropylsilane layer on Si/SiO2 substrates using ethanol as APTES solvent. | |
| MethodsX, 2020. 7: p. 100931. | |
| Jarvas, G., et al., Modification of hemodialysis membranes for efficient circulating | 13. |
| tumor cell capture for cancer therapy. Molecules, 2021. 26(16). | |

1. A kit of parts for assembly of a device for extracorporeal capturing of pathogenic material from a patient's blood thereby cleansing the patient's blood.

2. The kit of parts according to claim 1 said kit of parts comprising
beads, each bead having the diameter of 300 μm to 500 μm, preferably 350 μm to 450 μm and having or being suitable to have, attached to their surface, capturing molecules to capture (capable of binding) a pathogenic material,
a fillable cartridge, said cartridge having a first opening and a second opening and a first mesh and a second mesh to withhold the beads within the inner space of the cartridge,
means to allow the filling of the cartridge with beads.

3. The kit of parts according to claim 2 said kit of parts comprising
a set of one or more multitudes of beads, each bead having the diameter of 300 μm to 500 μm, preferably 350 μm to 450 μm and having, attached to their surface, capturing molecules to bind (capable of binding) a pathogenic material,
wherein beads of a multitude comprises beads having binding molecules for the same pathogenic material, and
different multitudes comprise beads having binding molecules for the different pathogenic materials
a fillable cartridge, said cartridge having a first opening and a second opening and a first mesh and a second mesh to withhold the beads within the inner space of the cartridge,
means to allow the filling of the cartridge with beads.

4. The kit of parts according to claim 2 said kit of parts comprising at least two multitudes of beads.

5. The kit of parts according to claim 1 said kit comprises
a multitude of beads, each bead having the diameter of 300 μm to 500 μm, preferably 350 μm to 450 μm and having, attached to their surface, capturing molecules to bind or capable of binding a pathogenic material,
a fillable cartridge, said cartridge having a first opening and a second opening and a first mesh and a second mesh to withhold the beads within the inner space of the cartridge,
means to allow the filling of the cartridge with beads,
a set of one or more (preferably at least two) multitudes of binding molecules for binding pathogenic material, wherein said binding molecules are useful to be attached to the surface of the beads,
wherein capturing molecules of a multitude (population) comprises beads having capturing molecules for the same pathogenic material, and different multitudes (populations) comprise beads having capturing molecules for the different pathogenic materials.

6. The kit of parts according to claim 5 said kit of parts comprising at least two multitudes of capturing molecules.

7. The kit of parts according to claim 1 wherein the cartridge has a first opening (1a) and a second opening (1b), a first mesh (2a) and a second mesh (2b) having pore size smaller than the diameter of the beads, as well as a useful space (6) of the cartridge which contains or is formed to contain the beads.

8. The kit of parts of claim 1 being part of a system having a control unit to control blood flow and preferably a pump.

9. The kit of claim 1 wherein the inner space of the cartridge the number of the specific binding sites is at least 50,000 per ml.

10. The kit of parts according to claim 1 having a volume of its useful space (i.e. wherein the beads are placed) of 20 to 500 ml, preferably 100 to 500 ml, preferably 300 to 400 ml in case of an adult human subject, preferably an adult human subject.

11. A cartridge prepared from the kit of parts according to claim 1 said kit comprising said beads and being useful for extracorporeal cleansing of blood.

12. A system comprising the cartridge according to claim 11 said system having a control unit and a pump to maintain the linear flow rate is preferably between 12 to 600 cm/min, preferably 12 to 300 cm/min, highly preferably 12 to 120 cm/min or 24 to 600 cm/min, preferably 24 to 300 cm/min, highly preferably 24 to 150 cm/min or 60 to 600 cm/min, preferably 60 to 300 cm/min, highly preferably 60 to 150 cm/min.

13. The system according to claim 12 wherein the linear flow rate is 24 to 150 cm/min.

14. The system of claim 12 wherein the inner space of the cartridge the number of the specific binding sites is at least 50,000 per ml.
